(19) United States
(12) Patent Application Publication
(10) Pub. No.: US 2014/0087412 A1
(43) Pub. Date: Mar. 27, 2014

(54) Method and Device for Application of Fluid Forces to Cells

(71) Applicants: Andreas Fouras, Clayton (AU); Gregory John Sheard, Clayton (AU); James Andrew Armitage, Clayton (AU); Michael David Curtis, Clayton (AU)

(72) Inventors: Andreas Fouras, Clayton (AU); Gregory John Sheard, Clayton (AU); James Andrew Armitage, Clayton (AU); Michael David Curtis, Clayton (AU)

(73) Assignee: 4DX PTY LTD, Clayton (AU)

(21) Appl. No.: 14/112,914

(22) PCT Filed: Apr. 20, 2012
(86) PCT No.: PCT/AU12/00405

§ 371 Date: Dec. 6, 2013

(30) Foreign Application Priority Data
Apr. 20, 2011 (AU) 2011901475

(51) Int. Cl. G01N 33/50

(52) U.S. Cl. CPC G01N 33/5091
USPC 435/29; 435/287.1; 422/505

(57) ABSTRACT

A system and method of determining biomechanical properties of a cell. A cell is introduced into a multiport flow device, the device being configured such that during fluid flow at least one stagnation zone arises in an expected location within the device. The cell is trapped in the stagnation zone of the device. A selected physical stimulus is applied to the cell, such as rotation, stretching or time-varying shear rate. The cell is observed while trapped to detect an absolute, differential and/or transient effect of the applied physical stimulus and to thereby determine biomechanical properties of the cell. Disease diagnosis may follow, by comparison to a normal control. Selectively directing the cell to a chosen outlet based on observed properties provides cell sorting, which may be implemented in parallel to increase throughput and/or in series to enlarge sorting criteria. Micro-particles may be investigated by use of appropriate particle model.

### TECHNICAL FIELD

The present invention relates to a microfluidics device and method for processing and/or assessing micro particles or objects, for example biological cells in a fluid, such as a fluid containing red blood cells. In part, the present invention provides a device and method for controllably applying physical forces such as shear and/or rotation and/or pressure to trapped cells in order to determine the cell response to such physical inputs, and detect changes or abnormalities in the cell response.

### BACKGROUND OF THE INVENTION

Cells are exposed to fluid forces in a variety of physiological contexts, including blood flow through arteries, capillaries and veins, air movement in lung alveoli and shear stress on endothelial cells and cardiomiocytes. For example, the relationship between shear forces and the health of endothelial cells has been widely investigated, and it has recently been shown that the effect of shear on platelets is critical for the proper understanding of platelet aggregation and thrombosis. In the case of erythrocytes (red blood cells: RBCs), links exist between the physical cell properties and a number of diseases, including malaria, some forms of cancer, dyslipidaemia, obesity and diabetes.

Cells are dynamic, exhibiting both rheological characteristics and active biochemical responses to applied force. The response of cells to externally-applied forces is complex and non-linear, due to interactions between membrane structure, internal organelles in the cell and viscous behaviour of the internal fluid, at least. Pathological conditions, such as diabetes, modify the biochemical properties of red blood cells, in turn changing the biomechanical response to physical forces. The malaria parasite modifies both the membrane chemistry and the internal structure of red blood cells, causing a dramatic increase in red cell stiffness. Environmental forces also provoke active cell responses, such as platelets activating, stem cells differentiating; blood vessel endothelial cells changing stiffness; and healthy cells becoming malignant.

Diabetes mellitus (hereafter simply diabetes) is the fastest growing chronic disease and the sixth leading cause of death in Australia, costing the Australian economy over 10 billion dollars per year. Worldwide, the number of adults with diabetes is projected to increase from 135 million in 1995 to 300 million in 2025. A range of complications occurs in diabetes, but micro-vascular disease is most often studied and those with diabetes have double the probability of developing vascular disease. There is strong evidence to suggest that increased blood viscosity and altered flow dynamics can contribute to diabetic disease, and decrease in the membrane distensibility of the RBC has been shown to precede vascular disease in diabetics. Importantly, there is also evidence that hyperglycaemia can change the properties of the blood cells that flow through these vessels, and that cells with such changed properties in turn contribute to the progression of vascular disease.

In young diabetic subjects, RBC distensibility is predicted by the level of RBC glycosylation and it is hypothesised that stiffening of erythrocytes is an important instigating factor in diabetic renal disease. Several factors may contribute to altered RBC distensibility in diabetes including an increased proportion of saturated fatty acids incorporated into membrane phospholipids and cholesterols. Saturated fatty acids pack tightly into a lipid membrane because there is low steric hindrance and high levels of membrane order resulting in a rigid, low fluidity membrane. It has been shown that apparently simple changes to the incorporation of particular fatty acids can significantly impact upon animal physiology, most likely due to increased membrane stiffness. Glycosylation (the chemical bonding of glucose molecules) of the cytoskeleton proteins further contributes to cell stiffness. There is also evidence that RBC intracellular adenosine-5′-triphosphate (ATP) is reduced in diabetes, resulting in an accumulation of intracellular calcium which can contribute to an increase in internal viscosity. Thus, stiffening of RBCs can be considered a precursory symptom of impending diabetic disease.

As another example, we turn to platelets. Pathological platelet function is intimately related to cardiovascular disease, which is the world's largest cause of death (including heart attack and stroke) and accounts for over 30% of all deaths in Australia, the USA and Europe. Shear forces applied to platelets have been shown to influence platelet aggregation and are thus relevant to thrombosis.

The effects of the malaria-inducing Plasmodium parasite serve as another example whereby red blood cell biomechanics are strongly related to disease. Malaria causes reduced deformability of RBCs. A key mechanism of fatality from malaria is blockage of capillaries caused by significantly reduced deformability of RBCs. Estimates of global death rates from Malaria vary from between 700,000 to 2 million annually. In addition, children who suffer from Malaria grow up with reduced intelligence, productivity and earning capacity.

To give another example of the importance of changes in the biomechanical and biophysical properties of cells and subcellular structures, it is noted that these properties influence, and are influenced by, the onset and progression of many forms of cancer.

Unfortunately, biomechanical and biophysical properties of cells and subcellular structures are difficult to assess in volume, efficiently, or even at all in some cases. Methodologies do exist to assess distensibility of individual RBCs and cells, but they are time consuming, non-standardized and highly variable. These methodologies include micropipette aspiration, atomic force microscopy (AFM), optical tweezers and optical stretchers, each of which provides a means of making some form of measurement of biomechanical cell response to an applied force, although only the former can provide a measure of cell viscosity. However the procedures are extremely delicate and time consuming, and require highly skilled practitioners. In the case of platelets even these advanced techniques can be too rough, and the measurement can itself cause unwanted platelet activation to occur. While precise measurements may be made on a single cell, making measurements on a very large number of cells in a short timeframe is not possible. Moreover, in the case of a sample of blood, in which only a small percentage of red blood cells have been affected by the early stages of a disease, it is impractical to apply these techniques to test the sample on a cell-by-cell basis as in even a small drop of blood the number of red blood cells numbers in the millions. Also these techniques are neither inexpensive nor portable, and are therefore generally unavailable to remote communities and developing nations. No device exists which is capable of non-invasive measurement of the elastic and viscoelastic properties of cells at a high rate of throughput. Consequently, to date relatively little work has been possible to investigate the relationships between disease type and severity on the one hand, and biomechanical and biophysical properties of cells and subcellular structures on the other hand.

It is also desirable to gain improved understanding of other forms of microparticles such as microdroplets of water suspended or coated in oil or the like.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

According to a first aspect the present invention provides a method of determining biomechanical properties of a cell, the method comprising:
introducing the cell into a multiport flow device, the device being configured such that during fluid flow at least one stagnation zone arises in an expected location within the device;
trapping the cell in the stagnation zone of the device;
applying a selected physical stimulus to the cell; and
observing the cell while trapped to detect an effect of the applied physical stimulus and to thereby determine biomechanical properties of the cell.

In some embodiments of the first aspect, the method may be used to diagnose a disease which affects biomechanical properties of the cell by detecting the presence of abnormal cells having abnormal biomechanical properties. In such embodiments, the cell may be a red blood cell and the biomechanical property may be the stiffness of the red blood cell as determined in response to the selected physical stimulus. In such embodiments the disease may be one of diabetes, cancer, obesity and malaria, and the cell may be an endothelial cell, a bone marrow cell, a cell from the lymphatic fluid or a cell from cerebrospinal fluid. In some embodiments of the first aspect, the method may be used to determine oocyte flexibility or stiffness, for example in order to assess oocyte viability during assisted reproductive technology (ART) such as IVF or for other purposes. Such embodiments thus provide an ability to measure the viscoelastic properties of oocytes before fertilisation takes place in order to select the oocytes with a superior likelihood of developing into a viable implantable embryo. Similarly, post-fertilisation characteristics of an embryo, such as flexibility, could be assessed. Of particular importance in such embodiments is the fact that the procedure is non-contact and will not damage the cell. With an added cell sorter as discussed elsewhere herein a screening tool for use in ART procedures may be provided.

According to a second aspect the present invention provides a method of sorting a cell, the method comprising:
introducing the cell into a multiport flow device, the device having at least first and second fluid outlets and the device being configured such that during fluid flow at least one stagnation zone arises in an expected location within the device;
trapping the cell in the stagnation zone of the device;
observing the cell while trapped to detect a property of the cell and, based on the observed property of the cell, selecting an outlet to which the cell should be directed; and
controlling fluid flow within the device to alter the location of the stagnation zone and to direct the cell to the selected outlet.

A method according to the second aspect, comprising a cascade of sorting stages each comprising a multiport flow device configured to sort cells based on detecting a unique property or property-set of the cell, such that the cascade of sorting stages may effect sophisticated multi-property sorting of cells.

Embodiments of the second aspect of the invention may thus sort a relatively large sample of cells in order to ascertain whether certain cells are present which can be indicative of a diseased state. For example, the cascade of sorting stages may be configured to separate out one or more of: cells which should not be present, cells of inappropriate maturity for the transport medium (e.g. immature white blood cells in blood which should still be in the bone marrow), cell abnormalities as disease markers, cells of a certain shape, cells of a certain size, cells which exhibit a certain fluorescence or spectral response, cells above or below a threshold for “activation” or biochemical response to mechanical inputs (mechanotransduction), platelets, platelets having a propensity to activate and/or aggregate, leukocytes, and/or cells having a certain stiffness. Each sub-group of cells separated out in this manner can then be further analysed to assist in diagnosis.

In embodiments of the second aspect, the observed property of the cell may be the response of the cell to a stimulus or to a therapy.

According to a third aspect the present invention provides a device for trapping a particle, the device comprising:
a chamber in fluid communication with at least two fluid inlets and at least two fluid outlets, the device being configured such that during fluid flow at least one stagnation zone arises in an expected location within the chamber, within which a particle may be captured and observed; and
the device comprising a means to introduce a component of rotational fluid flow within the chamber such that rotational forces can be applied to a particle captured within the stagnation zone.

The means to introduce a component of rotational fluid flow may comprise asymmetric device construction such that simply by causing fluid flow through the inlets and outlets gives rise to rotational flow about the stagnation zone. For example, the inlets and outlets may be asymmetrically positioned around the chamber such that flow from one inlet preferentially travels towards one outlet, exposing the stagnation zone to a rotational force. Additionally or alternatively, the means to introduce a component of rotational fluid flow within the chamber may comprise one or more microfluidic jets separate to the inlets and outlets, the or each jet being aligned such that rotation is induced in either a clockwise or anti-clockwise injection by causing fluid flow out of, or into, the or each jet.

In embodiments of the third aspect, the fluid controller is preferably further configured to control fluid flow through the inlets and outlets in a manner to apply stretching forces to a captured particle, for example by increasing inlet flow into the chamber through opposing inlets and providing reduced inlet flow through other inlets. In such embodiments, simultaneous rotation and stretching forces may thus be applied to a captured particle. Rotational forces may be imparted by use of asymmetrically configured ports such as an opposed flow four port device. Additionally or alternatively, rotational forces may be imparted by use of a device having more than four ports. For example the device may comprise 8 ports or more than 8 ports. It is further noted that an eight or more port device permits greater degrees of freedom in the physical stimulus which may be applied while retaining the sum of forces on the cell as zero in order to maintain the stagnation point, and for example permits simultaneous trapping, rotation and stretching of the cell, which in turn permits separate determination of internal viscosity and cell membrane stiffness from the cell measurements. In embodiments of the third aspect, each inlet and outlet may be positioned substantially within a single nominal plane, and with inlets being alternated with outlets around the perimeter of the chamber such that each inlet has an adjacent outlet.

In embodiments of the third aspect of the invention the particle may be a biological cell, a molecule, a micro-droplet or other such object of interest, or a group of cells or tissues. Where the particle is a cell, the third aspect of the invention may in some embodiments be utilised to investigate cell behaviour during tank treading (where the cell membrane rotates independently of the cell contents), tank treading being able to be induced by applying simultaneous stretching and rotational forces.

According to a fourth aspect the present invention provides a device for trapping a particle, the device comprising:
a chamber in fluid communication with at least two fluid inlets and at least two fluid outlets, each inlet and outlet being positioned substantially within a single nominal plane;
fluid controllers for controlling fluid flow through the inlets and outlets in order to form a stagnation zone within the chamber within which a particle may be captured and observed; and
at least one out-of-plane inlet or outlet whereby control of fluid flow through the out-of-plane inlet or outlet allows a captured particle to be moved towards or away from the nominal plane.

Embodiments of the fourth aspect may comprise more than one out-of-plane inlet or outlet. In such embodiments, suitable flow control through the out-of-plane fluid ports may be used to effect rotation of a captured particle about an in-plane axis. For example such embodiments may comprise four fluid ports positioned at the top of the channel and a further four ports positioned at the bottom of the channel, allowing rotation forces to be imparted in the rotation planes about the X and Y axes, for example to tumble the cell or to control an angular position of the cell about these axes.

The out-of-plane port(s) may be used to apply continuous flow for example to counteract a relatively buoyant or dense cell, or may apply impulsive flows to control z-position in a feedback system. Preferably, out-of-plane ports are provided in pairs to minimise the impact of z-flow on the x-y flow. Out-of-plane ports positioned on the z-axis both above and below the stagnation zone may be used to apply compression to a trapped cell in the z-axis, by simultaneously causing fluid flow into the device through the out-of-plane ports. Conversely, such ports may be used to cause stretching of the cell along the z axis by simultaneously causing fluid flow out of the device through the out-of-plane ports.

According to a fifth aspect, the present invention provides a method of characterising fluid conditions which cause platelet activation, the method comprising:
passing a platelet through a fluid path;
controllably altering flow rate and fluid pressure within the fluid path in order to subject the platelet to a shear rate which is time varying and which has a selected profile over time during passage of the platelet through the flow path; and
detecting whether the platelet activates in response to the shear rate profile experienced.

The time varying shear rate profile may be generated by varying flow rate through a simple through-flow channel of constant cross section, or may be similarly generated within a four or more port stagnation chamber, or within other device geometries. Embodiments of the fifth aspect of the invention may be used to explore the conditions under which platelets of a single subject are caused to activate, and to determine whether such conditions are normal or abnormal and whether the subject (e.g. a human or animal) has normal or abnormal platelet activity. Embodiments of the fifth aspect may additionally or alternatively lead to novel configurations of vascular prostheses and implants, which are designed to avoid generating such shear rate profiles which are known to cause platelet activation.

According to a sixth aspect, the present invention provides a method of determining biomechanical properties of a cell, the method comprising:
introducing the cell into a multiport flow device, the device being configured such that during fluid flow at least one stagnation zone arises in an expected location within the device;
trapping the cell in the stagnation zone of the device;
first observing the cell while trapped, to determine initial biomechanical properties of the cell;
after the first observing, applying a selected stimulus to the cell; and
after applying the stimulus, observing the cell a second time while trapped to determine subsequent biomechanical properties of the cell caused by the effect of the applied stimulus upon the cell.

The applied stimulus may be a physical stimulus such as stretching, rotating or a changed compression of the cell. In a preferred embodiment of the sixth aspect, the stimulus is a physical stimulus and the cell is observed during application of the stimulus in order to measure transient effects of the stimulus. Notably, in embodiments in which the initial biomechanical properties of the cell, the transient response of the cell to the physical stimulus, and the subsequent biomechanical properties of the cell are all obtained, allows both cell viscosity and membrane stiffness of red blood cells to be determined. It is particularly useful to obtain such information from red blood cells as the short life (approximately 110 days for humans) of red blood cells ensures that a current disease state is ascertained, unlike measurements obtained from other cells which live longer. Moreover, while knowledge of elevated membrane stiffness alone for example could be indicative of either late stage diabetes or malaria, obtaining the additional knowledge of internal cell viscosity gives the option of differentiating between diseases in two measurement dimensions and not only one. Preferred embodiments of the invention may further obtain a third measure of biomechanical cell properties, such as cell behaviour during tank treading (where the cell membrane rotates independently of the cell contents), tank treading being able to be induced by simultaneous stretching and rotational forces applied in accordance with the present invention. The cell's resistance to tank treading gives further information on cell structure. Preferably, measurements are obtained of one or more of the cell's membrane shear modulus, the membrane viscosity, and the apparent membrane bending stiffness, in order to avoid or reduce the likelihood of for example a diabetic being given a false positive result for malaria and a false negative result for diabetes.

In some embodiments of the sixth aspect of the invention an impulse or step change stimulus can be applied to the cell, through rapidly changing the inlet and/or outlet flow rates of the cross slot device. Additionally or alternatively, force functions of specific frequencies can be applied to the cell. Measurement of the cell response using high speed imaging or other suitable method can then be used to analyse frequency dependent properties of the cell. Such embodiments may be beneficial in permitting measurement and understanding of the structural modes of the cell membrane, as distinct from the internal cell contents, and may also be beneficial in enhancing understanding of the viscoelastic properties of the cell. Such embodiments may present a significant diagnostic benefit, in that a change in stiffness of the blood cells such as may be caused by diabetes can be diagnosed without the need to estimate the normal baseline stiffness of the cell for the cell age and age of the person from whom the blood was extracted. This is because disease states may show up as a change of the cell modes in response to a transient or fluctuating input, which can be far more specific than an overall stiffness measurement.

In further embodiments of the sixth aspect of the invention, the stimulus may be a chemical stimulus, whereby chemical changes to the carrier fluid can be effected and perfused through the cell membrane. In such embodiments, the cell is initially trapped, biomechanical cell properties are measured, and then the cell is perfused with a known compound (e.g. drug, phosphate buffered saline with raised ion-content), by the addition of factors to the fluid flow medium, after which the biomechanical properties of the cell are re-measured. Perfusion may occur while the cell is trapped in the saddle or may in some embodiments occur by controllably moving the cell away from the saddle point and into a region of high flow, then returning the cell to the saddle point. The change in cell properties can be used to determine the response of the cell to the drug, which may be useful pharmaceutically, or used to determine pathological conditions. For example, perfusing diabetic RBCs with glucose or calcium (Ca2+) ions. These drug delivery studies can be carried out in situ and in an automated fashion. Oxygen, carbon dioxide and other gaseous compound concentrations will also affect cell properties, and thus some embodiments may comprise changing the partial pressure of gasses and examining biomechanical properties of the cells of interest by observing cell responses to the changed partial pressures.

In some embodiments of the sixth aspect more than one stimulus may be successively applied to the cell, with biomechanical properties of the cell being determined during and/or after one or more or all of the stimuli, for example to investigate the effect of a series of stimuli upon the cell.

The stimulus may be a change in pressure or shear stress, applied by symmetrically increasing or decreasing the force on the cell so that it remains at the saddle point but experiences different loads. Cells could then be sorted on the basis of the type of force to which they were subjected, and their response thereto.

Observing the response of the cell to the stimulus may comprise collecting the buffer/fluid carrier, and analysing that fluid for the presence of substances released by the cells in response to a graded physical force or other stimulus. Such embodiments may be of particular use in observing the response of endothelial cells, however many epithelial, stromal neural cells and glial cells will also release substances in response to stimuli, permitting such observation. Thus, observing the cell response to the stimulus may comprise testing the fluid flow medium downstream from the stagnation zone in order to detect excretion of factors by the cell in response to the stimulus.

According to a seventh aspect, the present invention provides a method for diagnosing or assessing the stage of a disease or disorder in a subject, comprising:
obtaining a cell sample from the subject;
introducing a cell from the cell sample into a multiport flow device, the device being configured such that during fluid flow at least one stagnation zone arises in an expected location within the device;
trapping the cell in the stagnation zone of the device;
applying a selected physical stimulus to the cell;
observing the cell while trapped to detect an effect of the applied physical stimulus and to thereby determine a biomechanical property of the cell;
comparing the biomechanical property of the cell to a normal control of the same cell type as the cell sample from the subject to detect evidence of the disease or disorder.

According to an eighth aspect the present invention provides a method for diagnosing or assessing the stage of a disease or disorder in a subject, comprising:
obtaining a cell sample from the subject;
introducing a cell from the cell sample into a multiport flow device, the device being configured such that during fluid flow at least one stagnation zone arises in an expected location within the device;
trapping the cell in the stagnation zone of the device;
first observing the cell while trapped, to determine initial biomechanical properties of the cell;
after the first observing, applying a selected stimulus to the cell;
after applying the stimulus, observing the cell a second time while trapped to determine subsequent biomechanical properties of the cell caused by the effect of the applied stimulus upon the cell;
comparing the biomechanical properties of the cell to a normal control of the same cell type as the cell sample from the subject to detect evidence of the disease or disorder.

In embodiments of the seventh and eighth aspects of the invention, the biomechanical property(ies) may be selected from the group consisting of the shear modulus, the viscosity and the apparent bending stiffness of the cell's membrane. The cell sample may be selected from the group consisting of peripheral blood such as red blood cells, body fluid, and tissue such as breast, bladder, male reproductive system, female reproductive system, bone, pancreas, brain, skin, digestive tract and lung tissues. Alternative embodiments of the device of the present invention may be capable of measuring the stimulus responses of cell types including but not limited to platelets, leucocytes, epithelial cells, stromal cells, endothelial cells, isolated neuronal cells and glial cells. In some embodiments the present invention may be adapted to examine the properties of conglomerations/colonies of cells or the properties of whole organs or tissues. The disease or disorder may be diabetes, dyslipidaemia, obesity, cancer or malaria. The cell may be diagnostic of a pre-invasive cancer or an invasive cancer. The stimulus may be a chemical or physical stimulus.

Some embodiments of the present invention may further provide a method of monitoring a response to a therapy comprising performing the method of the seventh or eighth aspects on a cell sample from a subject to whom the therapy has been administered. Some embodiments of the present invention may further provide a method for monitoring a subject for a disease or disorder comprising performing the method of the seventh or eighth aspects on a cell sample from the subject. Some embodiments of the present invention may further provide a method for selecting a subject for a therapy directed to a disease or disorder, comprising performing the method of the seventh or eighth aspects on a cell sample from the subject. The therapy may be selected from the group comprising anti-neoplastic therapy, antibiotic therapy, prophylactic drugs, lifestyle modification, vaccine therapy, biologic therapy and anti-angiogenic therapy. The method may further comprise planning a course of further diagnostic testing and treatment.

According to a ninth aspect the present invention provides a method of determining properties of a micro-particle, the method comprising:
introducing the micro-particle into a multiport flow device, the device being configured such that during fluid flow at least one stagnation zone arises in an expected location within the device;
trapping the micro-particle in the stagnation zone of the device by simultaneously monitoring particle position and measuring and controlling the fluid flow field in the stagnation zone; and
observing at least one behaviour of the micro-particle while trapped and determining at least one property of the micro-particle from the or each observed behaviour by reference to a micro-particle model.

The flow field in the stagnation zone may be measured and controlled by using a computed interpolated fluid hydrodynamics simulation control process, or alternatively by the use of particle image velocimetry.

The micro-particle may be a cell, molecule, a group of cells or molecules, or a non-biological particle such as a fluid micro-droplet suspended in an alternative fluid. The present invention may be applied to assist destructive and non-destructive testing of small bio-structures such as lipid coated treatment agents.

According to a tenth aspect the present invention provides a microfluidics fluid flow control system comprising:
a deformable member defining a microfluidic passage, whereby deformation of the member alters a cross section of the passage and the flow resistance of the passage;
a pressure source in fluid communication with the deformable passage;
an actuator for controllably causing deformation of the deformable member so as to controllably occlude the passage;
an actuator controller which linearly actuates the actuator, the actuator controller operating in response to an input signal indicating a desired fluid flow through the passage and in response to a feedback signal representing an observed feedback variable.

The deformable member may comprise a deformable silicon tube, or may comprise a deformable microfluidics substrate material such as PDMS. Occlusion of the passage may involve a partial occlusion or a complete occlusion stopping all flow. A reversal of the pressure gradient will drive flow in the reverse direction. Providing the actuator externally of the passage relative to the deformable member provides improved biocompatibility and improved compatibility with high throughput screening, by avoiding actuator contact with fluid inside the passage and avoiding the necessity to seal the zone where piston, plunger or valve vane penetrates the passage.

The operator may comprise a piston or plunger configured to controllably press upon the deformable member so as to partly or completely occlude the fluid flow in the passage. The actuator is preferably the plunger or armature of a solenoid controlled by the actuator controller. Preferably the actuator is a voice coil actuator. Use of such actuators effects a high speed response to a request for changed flow rate, such speed being advantageous in certain microfluidics applications. Importantly, use of a solenoid ensures that the solenoid motion is directly coupled to tube occlusion, as there are no intervening parts such as levers or cams, improving the correlation between passage occlusion and solenoid control voltage.

The actuator may be in disconnected contact with the deformable member such that depressing the actuator occludes the passage and such that upon withdrawal of the actuator the fluid pressure and/or resilience of the deformable member itself cause reduced occlusion of the passage. Alternatively the actuator may be connected to the deformable member such that during withdrawal of the actuator the withdrawal force of the actuator itself assists to reduce occlusion of the passage, which may improve the speed of the system response to requests for increased fluid flow rate. The latter approach may also act to prevent separation of the actuator and the passage and as such will reduce the time for the actuator to re-establish contact with the passage, hence improving the response of the system to a subsequent request for a decrease in the flow rate.

The observed feedback variable may be an observed fluid flow rate through the passage. The fluid flow rate may be measured by a flow meter, and is preferably measured by an optical fluid flow meter such as a PIV fluid flow meter to effect biocompatibility of the fluid flow measurement system. Alternatively the observed feedback variable may be the observed actuator position, as measured by an actuator position meter. The actuator position meter may be an optical range meter, or may comprise a spring and load cell configured such that spring force on the load cell is indicative of actuator position. Alternatively the observed feedback variable may be an observed pressure drop across the occlusion from which flow can be very accurately inferred. In preferred embodiments the actuator controller operates in response to feedback signals conveying more than one such feedback variable.

The passage defined by the deformable member is microfluidic in the sense that a cross-sectional area of the passage when not occluded is less than about one square millimetre. For example for a circular silicon tube the inner diameter of the tube could be about 1 mm. For embodiments applied to a deformable PDMS microchannel one of the passage cross sectional dimensions could be about 300 microns.

The pressure source preferably comprises a pressure vessel fed by a pressure pump for providing bulk pressure supply to the pressure vessel, a pressure sensor monitoring pressure in the pressure vessel, and a pressure sink for controllably reducing pressure in the pressure vessel. The pressure sink may comprise a bleed valve when operation is only required at or above ambient pressure, or may comprise a source of negative differential pressure or suction if operation is additionally or alternatively required at or below ambient pressure, or if improved response speed is required in response to requests for reduced source pressure.

The pressure pump for providing bulk pressure supply to the pressure vessel may in some embodiments be a fan. Similarly, the source of negative differential pressure may comprise a fan. Preferably, a valve is provided between the pressure pump and the pressure vessel to permit isolation of the pressure vessel from the pressure pump to improve the speed of the system response to requests for reduced pressure. Similarly, a valve is preferably provided between the pressure vessel and the source of negative differential pressure so as to permit isolation of the pressure vessel from the negative pressure source and to improve the speed of the system response to requests for increased pressure.

To accommodate processing of small sample fluid volumes, such as microliters of blood, the pressure vessel is preferably charged with a carrier fluid, whether liquid or gas, which is arranged to be in pressure communication with the sample fluid of interest. For example where the carrier fluid is a gas the gas may be exposed to the sample fluid in a sample fluid reservoir such that the sample fluid exits the reservoir at the controlled pressure. Alternatively a membrane or diaphragm may be provided to separate the carrier fluid and sample fluid while effecting pressure communication therebetween such that the sample fluid exits the reservoir at the controlled pressure.

Some embodiments of the tenth aspect may thus yield fast and stable microfluidic flow control. Embodiments of the tenth aspect may be applied to control fluid flow of a multiport device in accordance with any of the first through ninth aspects, or to provide fluid control for other types of microfluidic devices. Alternative embodiments of the tenth aspect may be applied to effect fluid flow in an artificial heart, air flow in an artificial lung, or to perfuse a body or organ with blood or a lung with air. In particular the improved response speed and control accuracy afforded by some embodiments of the tenth aspect may provide for such fluid flow to be controlled in a complex time varying profile which mimics the time varying profile of naturally occurring physiological fluid flows, such as the complex pulsatile human aortic flow waveform in the case of blood flow.

The tenth aspect further recognises that selection of a pressure source rather than a displacement pump is critical for microfluidics fluid flow control, in order to substantially decouple fluid pressure from flow rate.

According to an eleventh aspect the present invention provides a computing device configured to carry out the method of any of the preceding aspects of the invention. According to a twelfth aspect the present invention provides computer software for causing the method of any of the preceding aspects of the invention to be carried out. According to a thirteenth aspect the present invention provides a computer program product comprising computer program code means for carrying out the method of any of the preceding aspects of the invention.

Cells and fluids addressed by embodiments of the present invention may include any which respond to physical stimulus. For example platelets respond to changes in shear stress by undergoing changes in shape and membrane properties (activation, tethering, tether retraction, etc), and pressure and rotation may also affect platelet function. Red blood cells when diseased, such as those affected by malaria or cancer, have altered elasticity and exhibit an abnormal response to hemodynamic factors such as shear, rotation and pressure. White blood cells play a critical role in plaque formation in response to changes in hemodynamic and chemical environment. Other cells/fluids to which this invention may be applied may include endothelial cells, epithelial cells, bone marrow, oocytes, early blastocysts, lymphatic fluid, cerebrospinal fluid or tumour cells. In further embodiments, the device may be used to assess the biomechanical properties and motility of spermatozoa and when a sorting module is added, the device allows for sperm to be introduced into the cross slot, the biomechanical properties and ability to swim from the saddle point measured, and cells sorted on the basis of motility or cell distensibility.

“Biomechanical properties” as used herein includes biophysical cell properties, and could for example include RBC membrane distensibility or RBC intracellular viscosity. “Biomechanical properties” as used herein can also include biomechanical properties of subcellular structures which influence the biomechanical properties of the cell as a whole.

Reference herein to a “control device” or “controller” or the like includes control devices such as a microprocessor, microcontroller or firmware controller.

In embodiments of any of the above aspects, the physical stimulus may comprise a static or time varying stretching force applied by fluid control, a static or time varying rotational force about one or more axes, a static or time varying shear rate applied to the trapped cell, a static or time varying pressure for example arising from altered fluid pressure applied at the fluid inputs, or an acceleration caused by moving the stagnation point and trapped particle. The stimulus may comprise a simultaneous or sequential application of more than one such stimulus. The stimulus may be applied rapidly in order to elicit and observe transient cell responses in which cell viscosity dominates, or slowly to determine a steady state response of the cell dominated by membrane stiffness, or both in turn. The physical stimulus may be partly or wholly applied prior to trapping of the cell in the stagnation zone, for example being a velocity, acceleration or time varying shear rate profile applied as the trapped cell or particle approaches the stagnation point.

The present invention thus provides a device with the capacity to rapidly, sensitively, accurately and quantitatively measure the mechanical properties of individual micro-particles such as cells without physical contact. In some embodiments the biomechanical properties of hundreds of cells per hour may be measured, in contrast to manual approaches where 100 cells might take months to measure. Hence a statistical measure of an adequately large sample size of diseased cells can be produced quickly, efficiently and with a minimum of human input.

Preferably, the device is formed as a microfluidics device, thereby being miniaturised and allowing inexpensive and rapid operation and improved portability, for example in diagnosing one of the abovementioned diseases. Moreover, such microfluidics construction permits operation upon very small blood or fluid samples. Utilizing lab-chip technology may thus give high throughput capability. When combined with features such as sorting and dilution the device could become a home-use device for example to be used in concert with other self-monitoring devices such as blood glucose and glycosylated haemoglobin 1 c meters. Such devices embodying the invention may thus be of improved feasibility in remote applications, consumer (home-use) applications, general practitioner (GP) medical clinic benchtop use, or in the developing world.

Observing the captured cell or particle may comprise imaging by use of a camera or microscope or other imaging device. Alternatively 3D images may be acquired using holography (utilising phase information form a coherent light source) or 3D images acquired utilising the 3D point spread function of an objective. Shape and orientation of the captured cell or particle may be determined by automated edge detection analysis of images taken of the stagnation zone. Alternatively the 2D or 3D particle image may be compared with a computer model of said image to solve for one or more of cell size, shape, position, orientation or deformation. Preferably, such images are obtained at high speed during transient events experienced or applied to the cell or particle. For example, measuring a rate of stretching or deformation of the cell or particle may be conducted in this manner in order to measure cell velocity and acceleration. Imaging may determine cell position, cell velocity, cell acceleration, cell shape, cell orientation, and may be used to generate fluid control feedback to influence any of these factors. Imaging may be used to determine fluorescence such as two colour fluorescence (dye) or activation based fluorescence (natural chemical identification using for example calcium efflux or dyes), and/or may comprise spectroscopy. Imaging may be performed using one or more of any part of the imaging spectrum including any wavelength of light (including but not limited to IR, visible, UV, Xray), ultrasound, MRI or the like.

Preferred embodiments of the invention may thus find particular application in diagnosis of red blood cell (erythrocyte) disorders, including Malaria, diabetes, cancer, dyslipidaemia and obesity, which all affect the stiffness properties of red blood cells, or indeed in diagnosis of any disease or condition that results in alteration of the biomechanical properties (such as stiffness of internal viscosity) of any cell (such as diabetes, cancer or malaria). As another example, the fatty acid composition of cell membranes alters the flexibility of the cell membrane and incorporation of long chain fatty acids such as docosaheaenoic acid (DHA) into cells is associated with a favourable health benefit with regards to diabetes, hypertension and inflammatory disease. By measuring the cell flexibility (red blood cell or any other cell type) and determining how this changes with the consumption of a favourable DHA rich diet, one may be able to produce a diagnostic/predictive test for health benefits of fatty acids.

In some embodiments of the invention a cell trapping device may be used as a diagnostic device (disease, pathology, cell type detection) or as a sorter, and/or combined into one hybrid device. The combination of such devices (diagnostic, sorting, hybrid) into arrays dramatically increases the power and selectivity of the system. Such arrays of devices may be arranged in series for example to effect increased complexity of tasks performed such as multi-stage sorting, and/or may be arranged in parallel for example to increase speed and/or throughput. Embodiments which combine cell disease state classification and sorting may be used to effect dialysis or the like in order to remove diseased cells from a subject while returning non-diseased cells to the subject.

Embodiments of the present invention thus may provide for application of controlled and time varying shear rate and pressure forces to one or more cells, using a fluidics device. Trapping allows the complete optical measurement of the response of those cells to these forces, such as changes in calcium flux, cell-size and cell-shape, but using non-invasive imaging without physical contact with the cell which may corrupt the measurement. The cell response may be physical (e.g. stretching of red blood cells), biochemical (e.g. calcium flux due to activation platelets) or a combination of both (e.g. aggregation of platelets). It is to be appreciated that such detection of functional responses of cells and groups of cells will find a large number of uses, including but not limiting to measurement of red blood cells and platelets.

Notably, the present invention recognises that it is possible to simultaneously control time varying forces applied to a single cell, while controlling/maintaining the cell position for the purposes of imaging the biomechanical response to the applied forces, while also maintaining relatively normal flow conditions around the cell.

This invention thus recognises that microfluidic devices, with similar length scales to blood vessels and other biological fluid channels, enable the construction of a customised biomimetic environment in the laboratory. By customising the channel geometry, and hence the fluidic environment of cells, forces can be applied to cells that mimic those encountered in vivo.

This invention further recognises that investigating dynamic behaviour, such as the response to varying forces applied to a cell in real time, requires active control. The use of the fluids to directly manipulate cells offers a preparation-free, biomimetic approach to providing such control with a low risk of cell damage. In addition, the use of direct fluid manipulation allows multiple, independent, fluid control systems to be densely integrated on a single chip as a cascaded sorting and/or diagnostic device.

The present invention further provides the benefit of cell-specific measures and detailed statistics of such measures, as opposed to a gross average measure of the response of a population of thousands or millions of cells.

Some embodiments of the invention may be used in conjunction with other techniques for the testing and sorting of cell properties, such as fluorescence-aided cell sorting (FACS) and/or spectrometry.

### BRIEF DESCRIPTION OF THE DRAWINGS

An example of the invention will now be described with reference to the accompanying drawings, in which:

FIG. 1a illustrates a hybrid cell stretching and sorting device 100 in accordance with an embodiment of the present invention; FIG. 1b shows stretching and sorting of cells using the microfluidics device of FIG. 1a;

FIG. 2a illustrates the computed interpolated fluid hydrodynamics (CIFH) control simulation process applied to effect cell trapping and positional control in this embodiment of the invention; FIG. 2b illustrates a viscoelastic cell model;

FIG. 3 illustrates the efficacy of the control system of FIG. 2 in controlling the position of the cell using fluid flow control;

FIG. 4 shows the steady state error norm as a function of various PID gains, with FIG. 4a showing a contour of the RMS error of the final 20 seconds of the simulation for each solution, and FIG. 4b shows the variation of the error with varying differential gains in both directions, with the proportional gains fixed;

FIG. 5 shows the simulated results of accuracy of cell sorting using the cross geometry device of FIG. 1;

FIG. 6 schematically illustrates the port configuration of an eight port device of an alternative embodiment of the invention;

FIG. 7 schematically illustrates the configuration of an opposed flow device of another embodiment of the invention;

FIGS. 8a and 8b are schematics of a control system implementing a complete fluid/structure model for monitoring both fluid flow field and particle position, in order to effect particle trapping and/or sorting;

FIG. 9 illustrates positional control accuracy achieved in one embodiment of the invention;

FIG. 10 is an exploded view of a voice coil actuator assembly of a fluid flow controller, with replaceable fluid tube, in accordance with another embodiment of the invention;

FIG. 11 is a cross sectional view of an 8 port device in accordance with another embodiment of the invention;

FIG. 12 is a cross sectional view of a 5 port device in accordance with yet another embodiment of the invention;

FIG. 13 is a cross sectional view of a 6 port device in accordance with still another embodiment of the invention;

FIG. 14 is a schematic of a 4 port device in accordance with another embodiment of the invention, having an outlet filter;

FIG. 15 is a schematic of a 4 port device in accordance with another embodiment of the invention, having a cell injection inlet;

FIG. 16 is a schematic of a 4 port device in accordance with another embodiment of the invention, having a cell extraction outlet;

FIG. 17 is a schematic illustrating the use of pre-computed simulations to produce lookup data to speed high throughput cell analysis;

FIGS. 18a and 18b illustrate a fluid flow supply system suitable for use with the controller of FIG. 8;

FIG. 19 illustrates plunger operation for the fluid controller;

FIG. 20 illustrates various applications of the device of FIG. 1; and

FIGS. 21a-d illustrate the voice coil actuator of a preferred embodiment of the invention.

### DESCRIPTION OF THE PREFERRED EMBODIMENTS

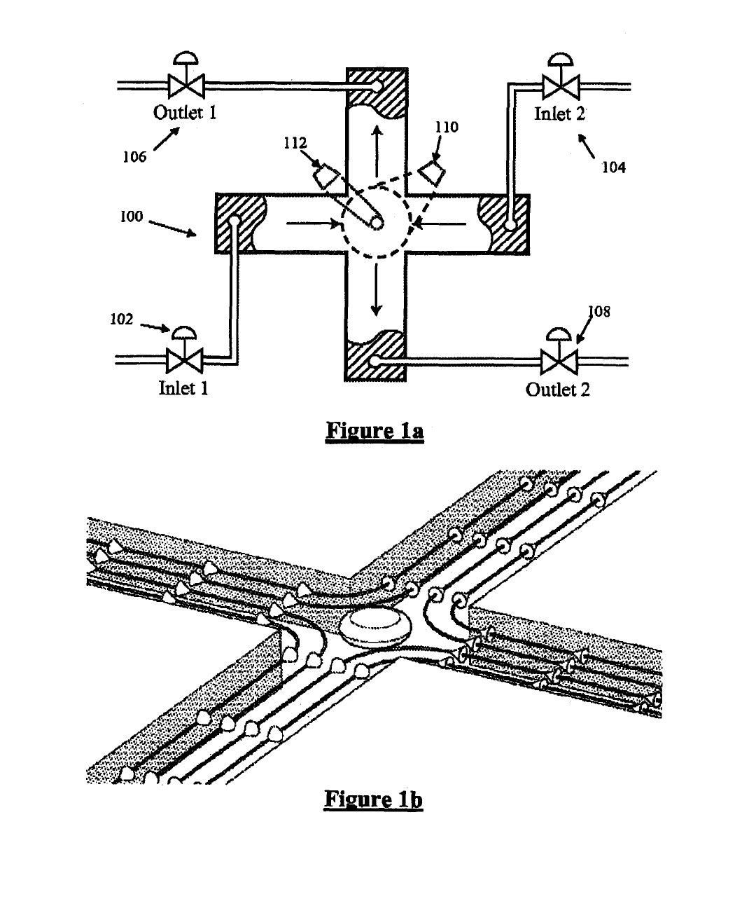

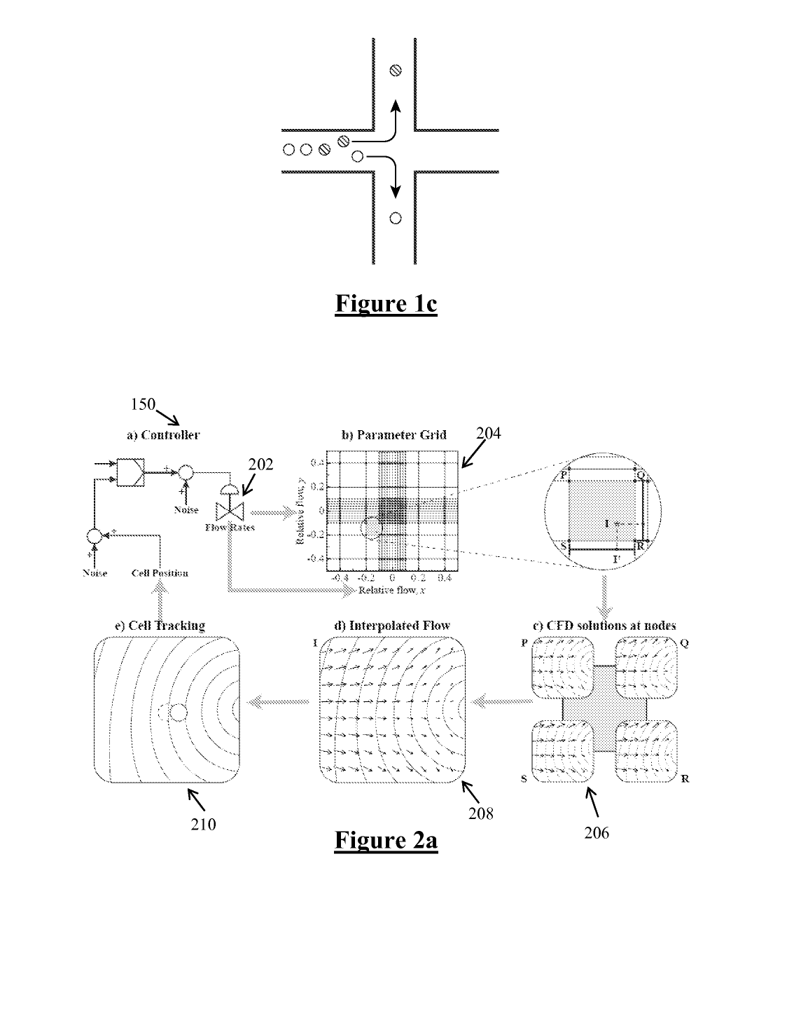

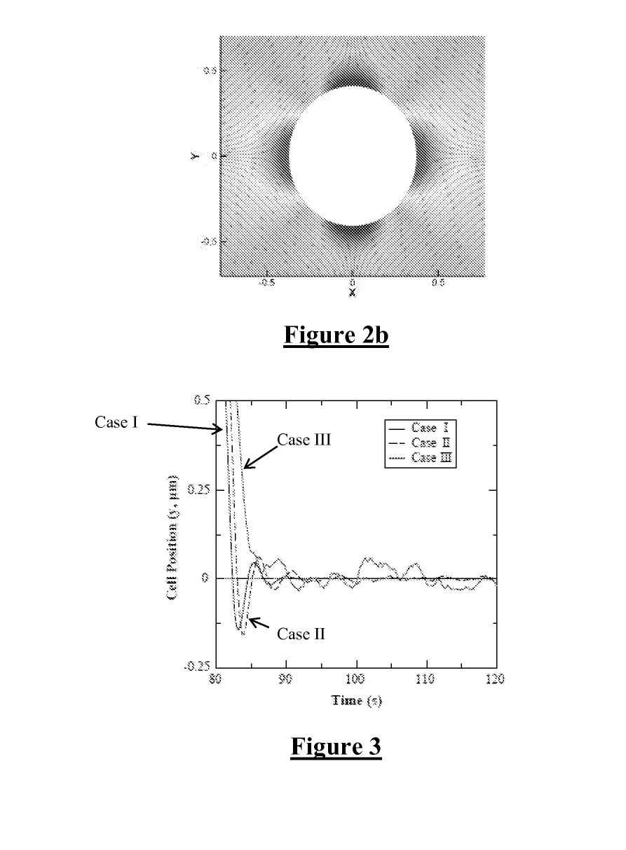

FIG. 1a illustrates a hybrid cell stretching and sorting device 100 in accordance with an embodiment of the present invention. Two inlets 102, 104 introduce fluid into the device 100, while two outlets 106, 108 receive fluid from the device 100. This flow configuration produces a stagnation point centrally within the device at which a cell may be trapped. That is, the twin opposing microfluidic jets create a saddle point flow, which is used to trap and stretch a particle, providing a gentle contactless environment. A first imaging device 110 is relatively widely focussed about the stagnation zone and is used to determine cell location, for use by a microprocessor based controller 150 (FIG. 2) in feedback control of the fluid flows to capture and then maintain the trap of the cell. A second imaging device 112 is focussed relatively tightly upon the stagnation zone so produced, to observe detailed characteristics and responses of a cell while trapped. After trapping concludes, the cell can be selectively directed to either one of the two outlets to effect sorting.

In more detail, the key components of the four-roll mill single cell shear chamber 100 are: (a) the cross-slot micro channel 100; (b) the cell position imaging system 110; (c) the high bandwidth fully controllable pumps/valves 102-108; (d) the cell shape imaging system 112; and (e) the control system 150 linking together components b-d. In this embodiment the micro channels 100 are fabricated from moulded PDMS, providing an accurate, relatively inexpensive surface that is chemically inert and mechanically robust as well as offering near-perfect optical access. PDMS also allows biocompatible coatings (e.g. Lipidure) to be applied either during or post manufacture which, due to the relative insensitivity of RBCs to altered chemistry, is of more relevance when processing other cells such as platelets. The fluid environment also allows a static level, or a time varying profile, of chemical conditions to be controlled, for example pH or oxygen levels. Collisions or interactions between cells or particles may also be induced and observed. When introducing a cell to the device through one of the inlets, suitable dilution control may be required in order to acquire just one cell. Alternative embodiments may be fabricated by etching silicon (SiO₂) on glass.

To allow stable trapping of RBC the cell position must be continuously monitored in real time. The most recent CMOS technology allows for cameras with moderate speed, sensitivity, resolution and throughput. This embodiment uses a monochrome camera 110 which operates at 200 frames per second with 640×480 resolution, while constantly streaming all image data to a gigabit Ethernet interface. This data stream is analysed in real-time by the control system 150.

In FIG. 1a it can be seen that the cell position can be controlled by the relative flow rate through the four valves shown (102-108), with the x position (left-right in the figure) being a strong function of the relative flow through inlets 1 and 2 and the y position (up-down in the figure) being a strong function of the flow through outlets 1 and 2. This embodiment therefore uses fully controllable high bandwidth biocompatible pumps 102-108, described further in the following. In addition, the shear chamber 100 requires a pump (not shown) to generate the pressure required to drive the fluid flow. Biocompatibility of the pumps is achieved by isolating the valves 102-108 from the fluid by placing them externally to biocompatible tubing.

In more detail, each pump consists of a pressure source, a flow meter, a hydrodynamic resistor and a control system. The pump ideally should not result in positive displacement, i.e. if resistance is infinite and flow is zero, pressure should not build up in the system. A simple fan type pump suffices. The flow meter constantly records the flow and reports to the control system. The ‘flow meter’ may be an appropriate combination of an imaging device and software, differential pressure transducer, ultrasonic transmission time sensor or other suitable monitor. The hydrodynamic resistor is a fast acting device to change the flow from the system under the action of the pressure source and under the control of the control system, in this embodiment comprising a computer controlled valve. The control system adjusts the resistor, and if necessary also the pump. In this design the resistor gives fine control of flow magnitude and timing, while the pump gives gross, slower acting, control, for example to reverse the flow direction entirely. The controller comprises a PID controller embedded in a microprocessor.

An alternative pump design could consist of a vessel, pump, pressure sensor, bleed valve and controller. In this embodiment the pump constantly supplies air to the vessel and the pressure is measured by the pressure sensor and relayed to the controller. The controller then appropriately actuates the bleed valve to maintain the pressure at the set level. This level may vary over time for the purposes described elsewhere herein. In addition a further valve may be placed between the pump and pressure vessel and also actuated by the controller increasing the accuracy, stability and pressure range of the system. The system may operate at either above or below atmospheric pressure, resulting in a change of direction to drive the flow. This design could isolate all biological components from all moving parts such as rotors, valve and pumps, granting ready biocompatibility.

Cell-shape data is acquired by a camera 112 having greater spatial resolution and increased magnification. In this embodiment camera 112 provides frame rates over 1000 fps at full frame (1 MP). Alternative embodiments may use larger and faster cameras if computing resources permit or if real-time analysis is not required. Such data acquisitions can be triggered by the control system 150 in synchrony with particular flow events which are in turn triggered at set intervals from successful trapping of the cell as determined by the control system 150 and cell-position imaging system 110. The response of the cell to the forces and hence membrane stiffness and internal viscosity can then be calculated at a later time.

In this embodiment cell-position image data from camera 110 is streamed in real time to image processing hardware, in this case a graphical processing unit (GPU) server. Here the image data is reduced to a cell centroid position and the cell's velocity and acceleration are inferred. This data can then be processed in software using a control algorithm and the cell position thereby controlled by manipulating the fluid flow rates into and out of each port of the device, as discussed further in the following with reference to FIG. 2. In alternative embodiments of the invention, camera 110 and camera 112 may be the same camera, with the images captured by the single camera being analysed in two different ways for the above-described purposes.

FIG. 1b shows stretching and sorting of cells using the microfluidics device 100. In FIG. 1b a red blood cell is shown (not to scale) undergoing stretching due to extensional flow. Fluid velocity, as indicated by the streamlines, results in shear stress on the cell, causing it to stretch. Sorting can also be achieved using this device by directing cells toward one of the two outlets in response to a detected characteristic of each cell observed. Control of the shear, saddle point position and therefore position of the cell in the microfluidic cross 100 can be effected by changing the relative flow rates of the inlets and outlets. For example, in FIG. 1a a higher relative flow rate through inlet 1 will result in the saddle point moving to the right, relative to the centre of the channel. Notably, shear rate can be adjusted by modifying the overall flow rate, and can be caused to follow a time varying profile, without requiring any change in the hardware geometry.

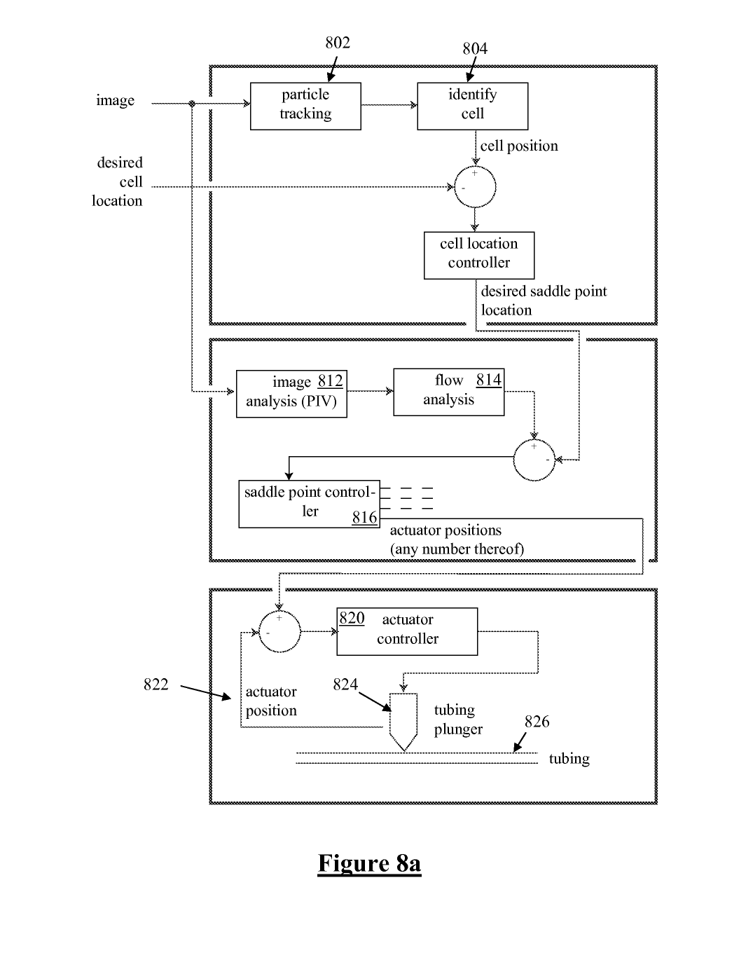

To provide a higher level of control of the cell (or particle) position, and as illustrated in FIG. 8a, the flow field in the stagnation zone may be measured and controlled directly. This occurs in parallel with the measurement of the cell (or particle) position (at 802, 804), allowing a greater level of robustness of the control system. This approach involves the use of particle image velocimetry (known as PIV). In the PIV step (at 812) the flow field is determined in local subregions of the image, through cross correlation analysis of consecutive frames of subregions. The location of the stagnation point may then be determined directly or through a process whereby the flow field is reduced in complexity (at 814) by a fit to a polynomial or other mathematical description. Thus a low order fit of the flow field is used to determine the flow saddle point. The location of the stagnation point or saddle is then deduced to be the location where the mathematical description shows zero flow. In the case where the local flow is known, and especially when the location of the flow stagnation point is known this can be used to control the cell position with greater precision and reduced response time. Such a control system could then request flow rates into the chamber in a nested manner, as follows.

Two control loops generally cannot directly control the same variable at once. However multiple loops can improve the performance of the system through nesting. The nested control in this case is as follows. The primary controller 816 seeks to control the saddle point and move it to a desired location. The controller 816 does so by signalling to the pumps to change to a certain flow-rate configuration, i.e. flow rates a, b, c & d for the 4 pumps respectively, output as desired actuator positions derived from the desired flow rate by reference to a lookup table (discussed further herein in relation to FIG. 17). The respective pump controller 820 for each flow controller then uses a lower level (nested) control system to achieve those flow rates. The respective pump controller 820 does so by asking the respective valve/plunger 824 to move to a certain position to effect a certain amount of occlusion of the associated fluid flow in the respective tube 826. The voice coil controllers then use a lower level (nested) control system to achieve that plunger position by adjusting the drive voltage.

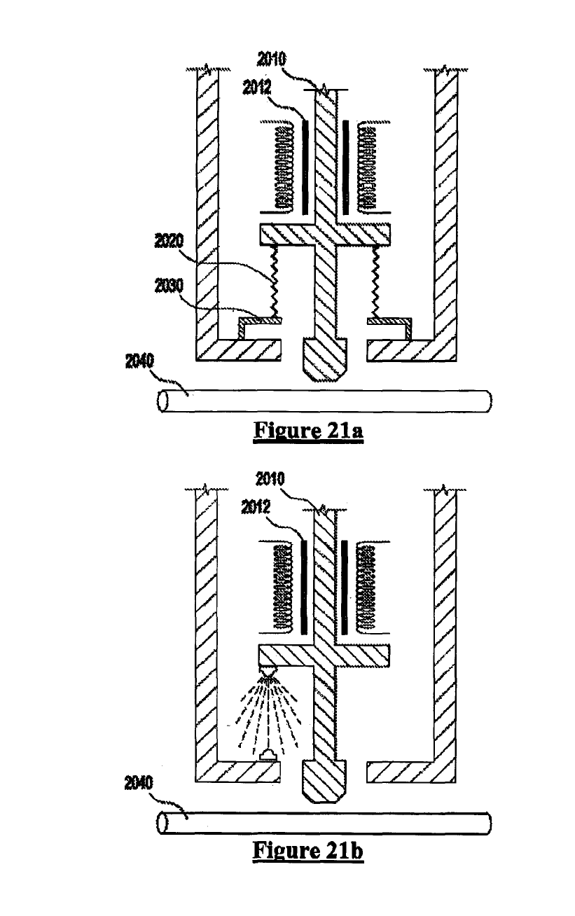

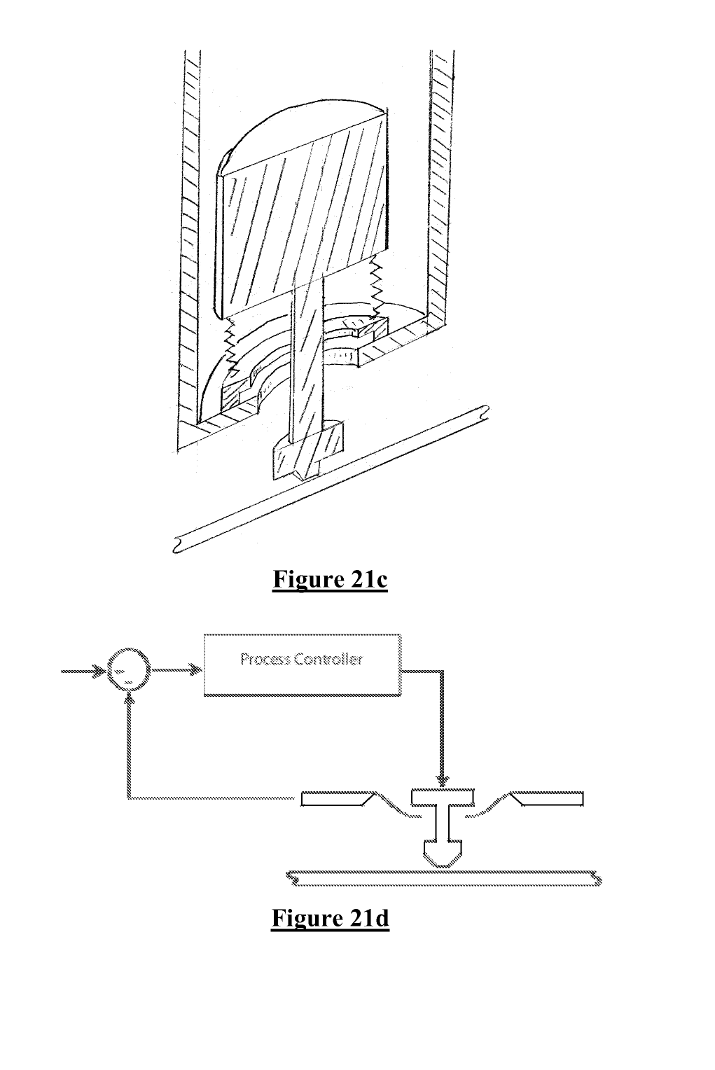

The system of FIG. 8a thus uses imaging as a feedback mechanism (at 802 and 812) rather than using a flow meter. Moreover, as indicated at 822, this device utilises feedback of the valve position itself as an additional control mechanism, and for example one possible mechanism to measure valve position is to preload the plunger with a spring and measure the spring force using a calibration strain gauge, as discussed further herein with reference to FIG. 21. Further, the system of FIG. 8a permits the pinch valve to be securely locked to a base mechanism allowing quick and easy replacement of tubes by the user thereby facilitating high throughput and repeated use, and provides a design that can easily be adapted to different tubing sizes.

FIG. 8b illustrates the nested control system of FIG. 8a as applied to the device 100 of FIG. 1.

The embodiment shown in FIGS. 8a and 8b implements a complete fluid/structure model, as follows. The algorithm employs an immersed boundary method for conducting three-dimensional simulations of one or more cells in a fluid flow. A front-tracking method (Tryggvason, G. & Unverdi, S. O., “A front-tracking method for viscous, incompressible, multi-fluid flows,” Journal of Computational Physics 100 (1), 25-37, 1992) is followed which allows the computation of the transient response of a cell that has a viscosity and a density differing from the surrounding fluid. The fluid flow is resolved using standard finite differencing methods on a fixed Cartesian grid. The cell uses a moving (Lagrangian) grid and the cell membrane is modeled as an elastic continuum using the constitutive equation developed by Skalak (1973) for blood cells.

In the above described embodiments, the observations of cell responses to stimuli are not always simple to relate to the underlying cell characteristics which lead to that response. For example, it is difficult to ascertain the cell membrane stiffness from a measurement of a change in elongation of a cell in response to changed fluid flows, both because the actual force applied to the cell by a given fluid flow is difficult to ascertain in a complex flow environment, and because linear models of a cell, for example modelling the cell as a spring and a damper, are too simplistic to permit a precise determination of the true cell properties.

FIGS. 21a-d illustrate the voice coil actuator of preferred embodiments of the invention; Position feedback is provided by a spring 2020 pressing on a load cell 2030 to a degree which depends on the actual actuator position as it depresses onto or withdraws from tube 2040. FIG. 21b shows a variant in which optical positioning detection is achieved by a light source casting a well formed cone of light, so that the light intensity detected by the light detector can be accurately and reliably converted to range and thus actuator position. FIG. 21c is a perspective cross-sectional view of the embodiment of FIG. 21a. FIG. 21d is a simplified representation of the embodiment of FIG. 21a, showing the plunger 2010 with a load cell either side. The control loop for controlling the plunger position is shown.

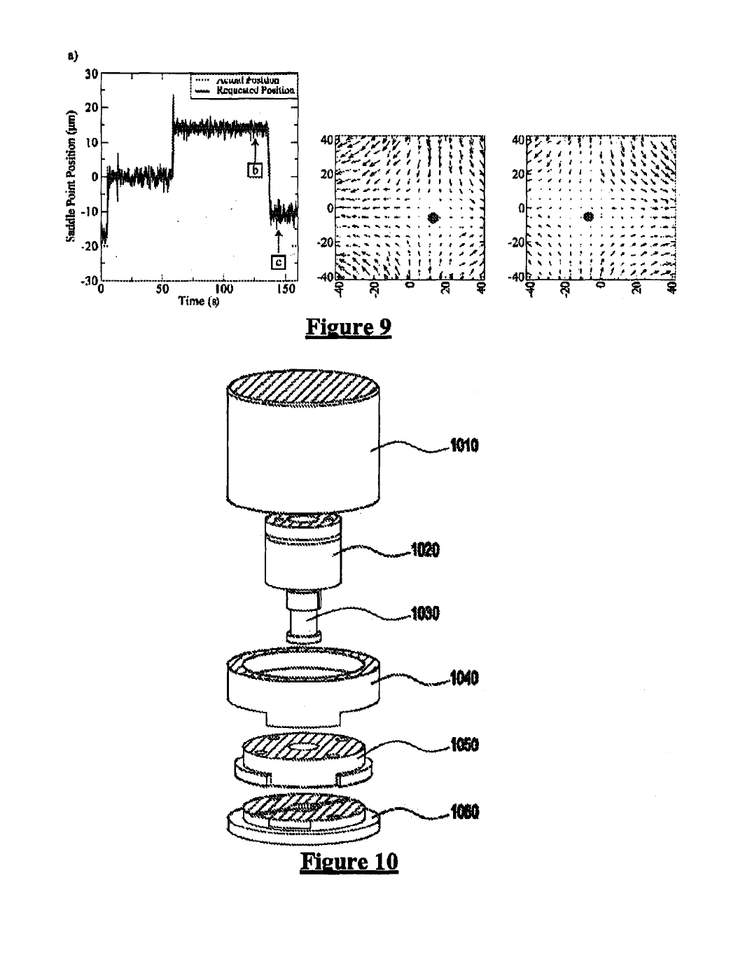

FIG. 10 is an exploded view of a voice coil actuator assembly of a fluid flow controller, with replaceable fluid tube, in accordance with another embodiment of the invention. The assembly comprises an exterior housing upper component 1010, an electromagnet/permanent magnet pair 1020, a plunger and sleeve bearing 1030, a locking collar 1040, an exterior housing lower component 1050 and a base and tubing guide 1060 including a mating bayonet lock. Notably, the base and tubing guide 1060 is configured to capture a microfluidics tube in a controlled and repeatable way, whereby the tubing lies in a slot that runs along the base and tubing guide. Capturing the tube into a known location prevents unwanted motion of the tubing during compression and better guarantees tube/plunger alignment and repeatable effects of plunger compression.

With reference to FIG. 17, when measurements of a cell response to certain fluid conditions within the chamber are obtained, they are compared to results of the computer model of a cell under the same fluidic conditions (i.e. the same input flow rates and chamber geometry). The cell properties in that model are varied (across a multidimensional space including at least membrane stiffness and intracellular viscosity). When the cell behaviour is matched in the simulation and in the measurement device then the properties of the cell in the device are deduced to be the same as those in the matching simulation. In more detail, computer generated modelling performed at 1702 executes simulations for a wide variety all parameters including input conditions, chamber environments and geometries and flow fields, and generates a multidimensional lookup table data 1704. Simulation resolution should be fine enough that interpolation of the desired order is accurate. To perform such simulations for all combinations of possible operational parameters at sufficiently fine resolution is computationally intensive, and currently prohibitively so for real-time operation of a computationally constrained high throughput system. However, once the multidimensional lookup tables are generated, it is a far simpler and faster computational step to query the lookup table using the measured characteristics (1706) of a trapped cell under observation, for example the observed cell elongation in response to a given geometry and flow rate and stimulus, the cell dimensions and/or the dynamic cell response (relaxation time or resonance). The underlying cell biomechanic properties which necessarily produce such an elongation response can then simply be read out (1708) from the multidimensional lookup tables 1704. Interpolation may be applied to the lookup table data depending on the fineness of resolution of the simulation values. The biomechanical properties read out of the multidimensional lookup table include elasticity values and viscosity. It is noted that viscosity has relatively minimal effect on final elongation as compared to elasticity, but that both elasticity and viscosity significantly affect cell relaxation time (or the cell resonant frequency in response to an oscillating stimulus), so that cell measurements 1706 preferably include at least a static elongation measurement and a dynamic relaxation or resonance measurement. Simulated parameters read out of the lookup table 1704 may further include cell volume, cell type and the like. Where the device 100 is intended for use in relation to one cell type only, the lookup tables may be reduced in size by only including lookup data for that cell type or particle category.

This embodiment thus provides a quantitative system for the high throughput measurement of cell mechanics through the complete integration of computer modeling with the device.

FIG. 18 is a schematic illustrating the valve, pump and actuator system of one embodiment, this being one option for implementation of the flow control system of FIG. 8. A desired flow rate is achieved in microchannel 1850 by producing a constant base flow using an air pressure system and providing finer control of this flow with a feedback controlled flow control valve 1840 of the type shown in FIGS. 8 and 21. Pressure levels are sustained through an air pump 1802 or gas bottle or the like, pressure box 1806, pressure sensor 1808, outlet valve (not shown) and a bleed valve 1810. The fluid flow control system of FIG. 18 provides an efficient yet highly effective manner of controlling fluid flow to a single port of a microfluidics device, including but not limited to any input or output of the devices of FIG. 1, 6, 7, 11, 12, 13, 14, 15, 16 or 20. The system 1800 is based on 2 fluids which in this embodiment are air and liquid, the latter being the sample fluid of interest. Alternative embodiments may use 2 liquids or a different gas than air, such as carbogen comprising approximately 90% oxygen (to oxygenate cells)+10% CO2 (to retain pH). Fan 1802 operates to pressurise gas with valve 1804 controlling, and selectively disconnecting, this flow into the pressure vessel 1806. The pressure in the vessel 1806 is monitored by pressure sensor 1808, and used by a controller (PID processor) to control the pressure via bleed valve 1820 as required. This configuration allows pressure control over a wide range, and in excess of the range typically provided by either valve 1804 or 1810 alone, effectively giving a multiplicative effect of the valve ability.

Pressure is directly shared from reservoir 1806 into the fluid reservoir 1804. Thus, a very tightly controlled pressure maintained in reservoir 1806 is the driving force for fluid exiting chamber 1804 towards microchannel 1850. The sample fluid in 1804 can be recharged by a refill flow line (not shown). Chamber 1804 may have a membrane or diaphragm separating the fluids while conveying the pressure. A membrane is beneficial in allowing the vehicle fluid/treatment fluid/diagnosis fluid and so on to be swapped through the device. Fluid passes valve 1840 (discussed above) and enters microchannel 1850 where camera 1860 provides a visual flow measurement (PIV) for feedback control of plunger 1840, effecting tightly controlled and swiftly responsive fluid flow in channel 1850.

In an alternative embodiment (FIG. 18b) the pressure vessel 1806 could also provide negative differential pressure to channel 1850 by providing a suction pump 1812 below the bleed valve, as shown in FIG. 18(b).

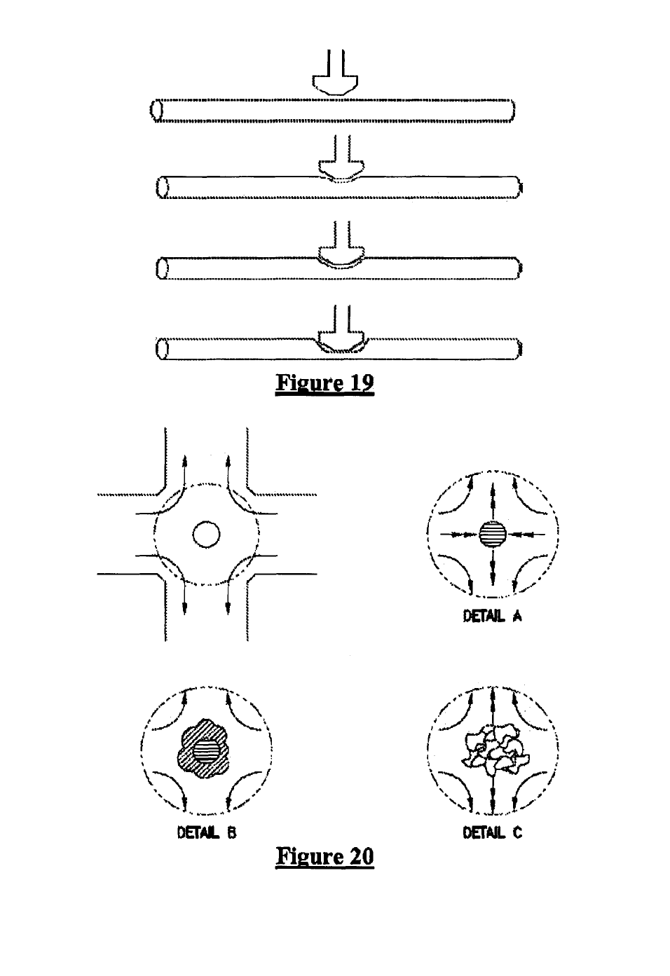

FIG. 19 illustrates plunger operation (descending). It is to be noted that the relation of plunger position to flow is non-linear, and varies with pressure, and so the control system should allow for this when determining a desired plunger position in response to a requested flow rate. It is desirable to provide nearby flow meters (e.g. PIV optical meters) to exploit flow rate control feedback.

All components which are in contact with blood (tubing, reservoir, flow probe) are disposable. The disposability eliminates blood clotting and enables faster cleaning between experimental runs. The formation of a clot is unacceptable as it would alter or destroy any thrombi formed inside the microchannel. Generally, large quantities of blood are unavailable for experimental use. By adopting a syringe-based reservoir, fluid consumption is kept to a minimum. Significantly, the adopted technique of FIG. 18 is adaptable and scalable for most physiological flow applications, for example from very low shear rates below the lowest shear rates typically arising physiologically, to very high shear rates at or exceeding the highest physiologically arising shear rates (for example in the heart).

The pump and control design of FIGS. 8 and 18 provides accurate real time flow control of steady, pulsatile and complex waveforms. This low impact flow control technique offers high adaptability for a wide range of frequencies and flow rates. The system includes a constant air pressure source to supply the base flow rate, a compact, disposable flow probe to the measure flow rate and a real time feedback control system to drive a fluid control valve. The fluid control valve allows for fine control over the base flow produced by the pressure source by pinching a silicone tube carrying the flow. The proposed flow control system offers high accuracy, the flexibility of producing physiological waveforms and the biocompatibility necessary for physiological blood experiments. The technique is highly scalable offering solutions to a wide range of flow control applications. These unique valves are currently prototyped with initial tests demonstrating the pump's capacity operating at a rate of over 300 Hz. Biocompatibility is achieved by isolating the valves from the medium by placing them externally to readily available biocompatible tubing. At present the pumps are fully controllable up to 300 Hz.

To measure the flow rate of the fluid in the system, a number of approaches can be used. Firstly, imaging can be used to measure the flow rate using a quantitative imaging technique, such as particle imaging velocimetry, particle tracking or optical flow. This method is inherently non-contact and lends itself well to biological flows as well as having very high bandwidth. Other non-contact techniques, such as ultrasonic transit time flow meters and laser Doppler interferometry may be suitable in some embodiments. However, the use of imaging to measure flow rate is advantageous in the preferred embodiment of this device as such imaging does not require additional hardware. In addition, ultrasonic transit time flow meters have a relatively low bandwidth which makes them undesirable for use in this application. It is recognised that other flow rate measuring devices could also be used. However, many of these require contact with the fluid and may be therefore unsuitable for and incompatible with biological fluids. In any case, the very low flow rates in microfluidic applications necessitate a new approach to flow measurement. A common type of flow meter used for low flows is the orifice plate flow meter. For the flow rates under consideration, these flow meters require very small orifice sizes in order to generate a measurable pressure drop, which in turn raises concerns about undue stresses on blood cells in high flow regions as well as introducing aggregation sites for thrombi.

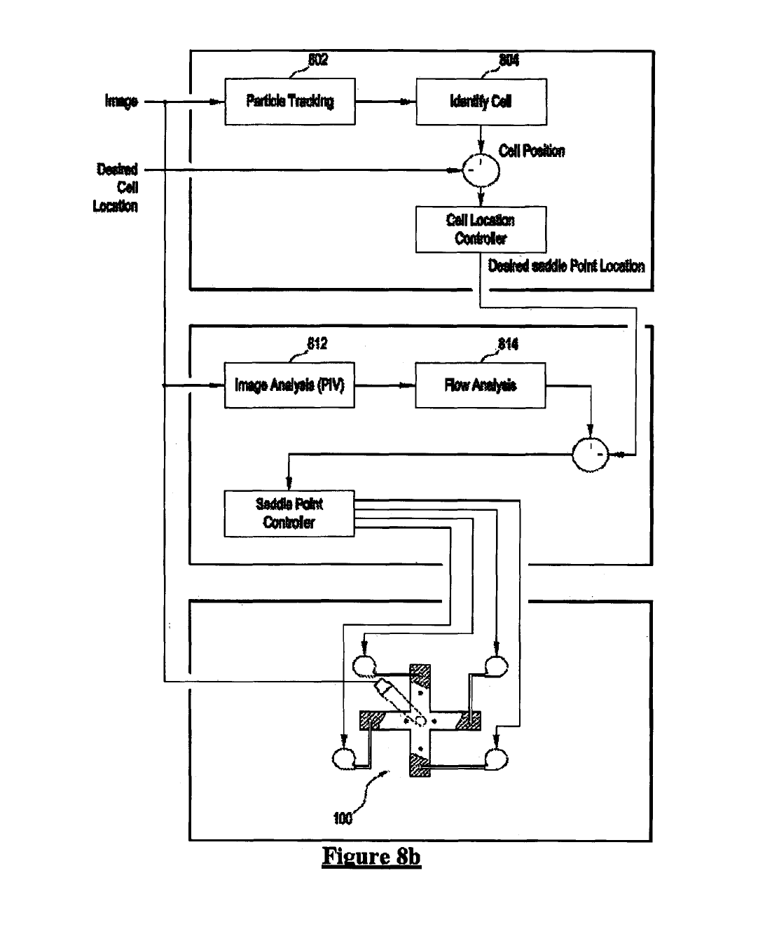

FIG. 9 demonstrates control of a RBC within the cross-slot microchannel device 100. In FIG. 9a the measured position of the flow saddle point is shown in response to a stepped input waveform for the requested saddle point by the controller. FIGS. 9b) and c) represent flow field maps of the central region of the cross slot for the time-points marked in (a). The flow saddle-point is marked by a dot to indicate where a RBC would be trapped. This figure demonstrates that fine control over the position of the cell while trapped is achieved.

This embodiment of the invention thus provides for using the device 100 to trap a cell, apply a known force to the cell before and/or during trapping, and simultaneously measuring the response of that cell while trapped, all in a lab-chip environment. By controlling the fluid forces while imaging the cell in real time, a complete or improved understanding of the cell's physical properties can be achieved. In the simplest sense: the elasticity of the cell can be obtained by dividing the extension of the cell by the extending force. Furthermore, by utilising lab-chip technology, other capabilities such as high throughput, and ancillary features such as sorting and dilution can be combined into a single device with capacity for miniaturisation.

It is noted that cell elasticity created by the complex geometry of the red blood cell and the inter-relationship between the membrane stiffness and the internal viscosity of the intracellular fluid introduces complexity to this arrangement. In addition, the fluid forces acting on the cell are obfuscated by the complex geometry of the cell and by the deformation of the cell under such forces, such deformation in turn altering the hydrodynamic response and the imparted forces, until a new equilibrium is reached. Accordingly, in this embodiment of the invention, these problems are addressed by utilisation of computer modelling. That is, in order to gain a thorough understanding of the precise magnitudes and distributions of stresses imposed on a cell by the device 100, computational modelling of the complete system is undertaken, including simulation of the flow field, the fluid-structure interaction between the fluid and the cell, and the mechanics of the cell itself. It is noted that such simulations can also inform the design and optimization process prior to fabrication of the device. The modelling also provides an important benchmark to assist in relating laboratory measurements of cell deformation to the actual stresses imposed by the device 100 on the cell, and hence permitting an accurate determination of the mechanical properties of the cell being tested and a more reliable comparison to other cells. In this embodiment the solver employed to compute the fluid flow within the device and around the trapped cell is a high-order computational fluid dynamics (CFD) code based on a spectral element method. Notably, spectral-element schemes offer outstanding spatial convergence properties and are computationally efficient. Of particular interest in the context of this invention, they are readily integrated with fluid-structure algorithms.

FIG. 2a illustrates the computed interpolated fluid hydrodynamics (CIFH) control simulation process applied to effect cell trapping and positional control in this embodiment of the invention. A controller 150 uses the cell position, determined from camera 111, as an input to determine a new set of boundary conditions, in this case relative flow rates indicated generally at 202. Available solutions for these boundary conditions are mapped to a parameter grid 204, which in this case is two dimensional as the z dimension can be neglected given the configuration of device 100. In the parameter grid 204 each dimension relates to a flow rate parameter (b). The point marked with an asterisk (*) in the exploded portion of the grid indicates the combination of flow rate parameters demanded by controller 150. The four nodes on the parameter grid forming the element boundary are identified (P,Q,R,S), where each node represents a complete CFD solution as shown at 206. The four solutions are then interpolated to find the resultant bilinear-interpolated flow at 208. Finally, hydrodynamic equations are used to compute the motion of the cell and hence the updated cell position, at 210.

A challenging component of this task is the development of a reliable cell mechanics model, and its incorporation into the code base understanding of microcirculation and the role that the extraordinary deformability of red blood cells plays in facilitating flow through capillaries with calibers far narrower than the RBC diameter. FIG. 2b illustrates a viscoelastic cell model (following Secomb, T. W., B. Styp-Rekowska, and A. R. Pries, Two-dimensional simulation of red blood cell deformation and lateral migration in microvessels. Annals of Biomedical Engineering, 2007. 35(5): p. 755-765), the contents of which are incorporated herein by reference, being useful in the CIFH simulation. This model is coupled with the CIFH fluid dynamics code to demonstrate the deformation of a cell when exposed to an extensional flow produced within device 100. Contours of pressure surrounding the cell are shown in FIG. 2b, along with velocity streamlines. Secomb et al's cell model comprises a two-dimensional set of interconnected viscoelastic elements, and good agreement between physical and modelled red blood cell characteristics is found when suitable parameters are assigned to the elements comprising the cell model.

Alternative embodiments of this invention may utilise the model of Peng, Z. L., R. J. Asaro, and Q. Zhu, Multiscale simulation of erythrocyte membranes. Physical Review E, 2010. 81(3), the contents of which are incorporated herein by reference. This is a more complex three-dimensional model constructed from a three-level multi-scale modelling approach in which the cell membrane is described at the complete cell scale as two continuum shells, the constitutive laws of the inner layer (the protein skeleton) are obtained from a molecular based model, and the mechanical properties of the spectrin (a key component of the skeleton) are obtained with a stress-strain model.

This approach provides an efficient control simulation technique, valid for typical microchannels, which is over 500 times faster than conventional time integration techniques. CIFH is a hybrid approach, utilising a combination of precomputed flows and hydrodynamic equations, and allows the efficient simulation of dynamic control systems for the transport of cells through micro-fluidic devices. The speed-ups achieved by using pre-computed CFD solutions mapped to an n-dimensional control parameter space, significantly accelerate the evaluation and improvement of control strategies and chip design. Here, control strategies for the microfluidic cross-slot, having a naturally unstable device geometry, have been simulated and optimal parameters have been found for proposed devices capable of trapping and sorting cells.

Investigating dynamic behaviour, such as the response to varying forces applied to a cell in real-time, requires active control. The use of the fluid flow to directly manipulate the cell offers a preparation-free, biomimetic approach with a low risk of cell damage, in contrast to alternative techniques using electric fields, magnetism, optical forces and surface acoustic waves. In addition, the use of direct fluid manipulation allows multiple, independent, fluid control systems to be densely integrated on a single chip. However, the interactions between actuators, the control algorithms, the cells under test and the dynamics of the fluid must be well understood in order to develop an efficient controller. Therefore, simulation of dynamic microfluidic systems is an important element in some embodiments of this invention.

The general approach to solving for solid body motion (e.g. a cell) within a fluid involves a computationally expensive iterative approach for each change in fluid boundary condition. The cell motion, flow field and reaction forces are computed in an iterative process until a solution which satisfies all coupled systems is found. This process must be repeated for every timestep. However, the present embodiment recognises that when the solid body can be described as a simple geometric shape, and the flow can be considered to be minimally perturbed by the presence and motion of the solid body, a simplified approach based on the hydrodynamic drag equations can be used effectively. Many cells can be approximated as spheres or ellipsoids and hence lend themselves well to this approach.

For this embodiment, a hybrid approach, using a combination of precomputed flows and hydrodynamic equations has been developed, allowing a dynamic control system for cells to be efficiently simulated. This simulation technique is used to characterise and design a control system to capture, trap and manipulate cells in the microfluidic cross slot 100.

There are four dynamic processes that must be modelled in order to simulate the motion of a cell in an actively controlled microfluidic environment: the change of the boundary conditions to the channel (for example input and output flow rates) by the control system 150; the change in fluid dynamics as a result of the these boundary conditions; the motion of the cell due to the dynamics of the fluid; and the perturbation of the fluid due to the motion of the cell. Most practical control systems can be defined deterministically as a function of the input conditions, in this case the motion of the cell in terms of position. Hence, the simulation problem is to solve for three unknown conditions: the fluid dynamics as a result of the varying input from the controller; the motion of the cell; and the flow perturbation. These three coupled systems can be reduced to a set of uncoupled systems, by imposing some modest constraints on the type of system and flow regime.

We consider first the fluid-structure coupling. In order to decouple the fluid flow calculation from the cell motion, some constraints are imposed on both the flow and cell. The flow field and the cell are a coupled system, with the motion of the cell having an effect on the flow field, and the resultant flow field feeding back into the motion of the cell. This two-way coupling requires, in the general case, iteration of the flow field and cell motion to evolve the system. However, for small Reynolds numbers in the regimes of creeping and Stokes flow (Re≲1), this coupling can be expressed in a closed form integro-differential equation known as the Bassett-Boussinesq-Oseen equation, which can be used to estimate the motion of a suspended particle in an ambient fluid. As the disturbance to the flow as a result of cell motion is incorporated in this equation, the disturbance field does not need to be calculated separately, reducing the numerical coupling between the base flow and cell motion to a one-way coupling. That is, the base flow can be computed independently of the cell motion.

As the disturbance field is calculated as part of the cell motion equation, not the fluid equations, these equations are most suitable for modelling a single cell in a flow. Where multiple cells are sufficiently close that their associated disturbance fields would interfere, accuracy decreases. In the general case, where the differences between the fluid velocity and the cell velocity are small and the densities of the cell and fluid are similar, the disturbance field reduces the base flow rate by 10% in the far field (5 diameters away from the cell). Practical requirements, such as the need to accurately identify and track individual cells, limit the maximum usable concentration. Hence the motion of moderate to low concentrations of cells can be approximated by an ensemble of single cells moving through a fluid with minimal loss in accuracy.

Next we consider flow field generation. Typically, in order to model the response to the changing fluid boundary conditions, a computational fluid dynamics (CFD) solver would be used to predict the flow field at every flow rate requested by the controller. This is computationally expensive and time consuming, especially when simulating a large number of control scenarios. Even for a two-parameter system, the number of CFD runs required would be large in order to fully cover the space. Instead, in the present approach, the space is sampled and an interpolation approach is used to evaluate the predicted flow field in between these sample locations. For any flow in the Stokes flow regime, the inertia terms in the Navier-Stokes equations are negligible, hence the flow field is a function only of the geometry (which is fixed) and the boundary conditions (which are time-varying). Therefore, the flow can be considered analogous to a Linear Time-Invariant (LTI) system, where the same flow field will be produced for a given set of inputs (that is, the set of boundary conditions), regardless of the time history of the system. The cell is considered to have a minimal impact on the flow field, as discussed previously, therefore not affecting the time invariant properties of the flow. It follows that, with sufficient sampling density, interpolation between the sampled flow fields can be applied to derive the flow field for any combination of boundary conditions.

Flow solutions are obtained over an n-dimensional grid of the input parameters. In this embodiment, a two dimensional grid 204 of flow rates is used. However, more complicated flow geometries may demand a grid with a larger number of dimensions. When a set of flow rates is demanded by the controller, these flow rates are mapped to the parameter space grid. In order to find the final flow field, a bilinear interpolation is performed between the four neighbouring CFD solutions (see P,Q,R,S in FIG. 2). This is similar to the bilinear interpolation scheme used in finite-element analysis, where the interpolation typically takes place between four individual scalars rather than four complete solutions. To reduce the computational overhead associated with interpolating four large meshes, the interpolations are evaluated using a lazy evaluation scheme, whereby the values at each node of the interpolated CFD solution are only computed when needed by the simulation code.

We next consider actuator modelling. A limitation of the above approach to modelling the change in boundary conditions is that it assumes that a change from one boundary condition to another can be undertaken instantaneously, regardless of the physical plausibility of this. However, for a non-turbulent, viscous flow, the response time of the fluid system will be dominated by the response time of the actuator controlling the fluid flow rate. Hence, fluid response time is incorporated with the actuator response time to give an effective system response parameter. In this embodiment, actuator response is modelled as a first order system, which will exponentially converge on a final value at a rate determined by the system time constant:

$$\frac{d q}{d t} = \frac{1}{τ} ( {q}_{fv} - q ) , \qquad ( 1 )$$

where q is the flow rate, q_{fv} is the final value of the flow rate (the demand flow rate) and τ is the time constant. While a simple first order model has been used, the actuator model can be extended in the general case to a discrete transfer function with higher order responses and incorporate non-linear behaviours such as actuator hysteresis.

A typical practical actuator is also limited by positioning resolution. The sources of error in an actuator may include digitisation resolution and noise; sensor accuracy and the response of the positioning controller 150. The limiting response of the controller 150 can be modelled as follows: (1) Digitisation noise and resolution are modelled by first quantising the demanded value to the actuator resolution, followed by the addition of zero mean white noise (FIG. 2), where the noise amplitude is equal to the half the resolution.

We now turn to cell tracking A fourth-order Runge-Kutta integration scheme is used to integrate the cell velocity and hence track the evolution of its position over time (FIG. 2, 210). An approach common in fluid visualisation is to assume that the cell is infinitesimal and integrate the massless, dimensionless equation, where the cell velocity is equal to the flow velocity. This approach does not fully capture the cell dynamics in this case as typical cells cannot be considered infinitesimal relative to the dimensions of a microchannel. Instead, the hydrodynamic equation for particle drag in a flow, the Bassett-Boussinesq-Oseen equation is integrated:

$${m}_{e} \frac{d v}{d t} = - \frac{1}{2} {m}_{f} \frac{d}{d t} ( v - u - \frac{{α}^{2}}{10} {∇}^{2} u ) - 6 {παμ}_{f} ( v - u - \frac{{α}^{2}}{6} {∇}^{2} u ) - \frac{6 {πα}^{2} {μ}_{f}}{\sqrt{{πμ}_{f}}} ( {\int }_{0}^{t} \frac{d}{d t} \frac{( v - u - \frac{{α}^{2}}{6} {∇}^{2} u )}{\sqrt{t - τ}} d τ ) + ( {m}_{c} - {m}_{f} ) g + {m}_{f} \frac{Du}{Dt} . \qquad ( 2 )$$

where m_{c} and m_{f} are the cell and fluid mass, respectively; μ_{f} is the fluid viscosity; α is the cell radius; v is the cell velocity vector; and u is the fluid velocity vector. The equation is reduced in complexity by taking into the account the following factors: the Faxen terms (∇²u) can be neglected when

$$\frac{α}{l} << 1 ,$$

where l is the channel length scale, a condition which holds except for very small channels or large cells; and, the integral term (known as the history term) approaches zero for Reynolds numbers less than 1. Additionally, this embodiment aims to model scenarios where the cell is already present in the flow and does not start from rest, hence reducing the importance of the history term in this case. For this analysis, buoyancy is ignored as only the in-plane velocity of the cell within the channel is of interest. Finally, the sphere drag used for the drag term in (2), i.e.

F=−6παμ_{f}(v−u) (3)

is substituted for the more general inertial drag equation

$${C}_{D} = \frac{2 F}{{ρ}_{f} {πα}^{2} {‖ v - u ‖}^{2}} , \qquad ( 4 )$$

This formulation is equivalent to that in (3), but lends itself more readily to describing aspherical shapes such as ellipsoids. For example, C_{D}=24/Re_{c} for drag on a sphere at low Reynolds numbers. Where the cell is known to be undergoing deformation due to fluid forces, the drag coefficient C_{D} can additionally be varied to simulate the change in hydrodynamic behaviour due to the shape change.

Hence, and expressing the masses in terms of areal density,

$$\frac{d v}{d t} = - \frac{1}{2} \frac{{ρ}_{f}}{{ρ}_{s}} \frac{d}{d t} ( v - u ) - \frac{1}{2} \frac{{ρ}_{f}}{{ρ}_{s}} {C}_{D} ‖ v - u ‖ ( v - u ) + \frac{{ρ}_{f}}{{ρ}_{s}} \frac{Du}{Dt} . \qquad ( 5 )$$

In this embodiment the behaviour of a limited observer is also modelled. Positional data into the controller will be limited by the resolution and update rate of the cell position sensor (in many cases, this would be a camera coupled with appropriate software). As there is little benefit in updating the controller parameters faster than input can be obtained from the sensors, the update rate of the sensors is modelled by adapting the controller update rate to be equal to or slower than the projected frame rate of the cell sensor. Resolution is modelled as a digitisation process, similar to the actuator model described previously, with the position obtained from the cell tracking model combined with a white noise process (FIG. 2, 150) and quantised to the imager resolution.

A simple control algorithm, based on PID (proportional integral derivative) control is used in this embodiment to control cell position (FIG. 2). While the technique is not limited to PID, this algorithm was found to produce adequate results for the control problems presented. Without loss of generality, the control algorithm can be defined in terms of a pair of independent one-dimensional PID algorithms (one for each axis of control −x and y)

e=xₓ−x_{c} (6)

f=K(e+K_{D}ė+K_{I}∫edt), (7)

where e is the error, xₛ and x_{c} are the desired and actual positions of the cell, respectively and K, K_{I} and K_{D} are the proportional, integral and differential gains, respectively.

Using the above modelling, a computational fluid dynamics (CFD) model of the cross slot geometry was constructed, with a nominal channel width of 100 μm. The Reynolds number, as defined by the channel width and the inlet flow velocity, was 1. The high-order spectral element solver VIPER was used to accurately compute the solutions to the flow. As stated in the preceding, a parameter space grid of the possible solutions is needed to efficiently generate flow fields for the control system. A non-dimensional parameter for the ratio of the flow velocities between the two opposing inlets was defined

$${f}_{x} = \frac{{v}_{1} - {v}_{2}}{{v}_{1} + {v}_{2}} , \qquad ( 8 )$$

where v₁ and v₂ are the average inlet flow velocities for inlets 1 and 2, respectively (see FIG. 1). The flow rate deviation can be defined similarly for the two opposing outlets. CFD solutions for over 350 combinations of inlet and outlet parameters were computed. The two dimensional location of the saddle point was used to map the change in the characteristics of the flow with respect to the input parameters. In each case, the saddle point position was identified using an automated Levenberg-Marquadt optimisation scheme, whereby a conic section was fitted to the velocity magnitude field in the approximate vicinity of the saddle point. Validation was performed using flow model and PIV (particle image velocimetry) software. Flow velocity fields show excellent agreement at a number of saddle point locations. The relationships between the relative inlet flows and saddle point for CFD and PIV showed good qualitative agreement. In both cases, the change in saddle point position and flow structures had a piecewise linear relationship, demonstrating that bilinear interpolation can be used to accurately obtain solutions for any combination of fₓ and f_{y} within the solution space (FIG. 2).

To assess trapping, using the CIFH method, the speed and repeatability of cell capture and the stability of a cell trap based on a cross-flow geometry can be assessed in a computationally efficient manner. Cells enter from one of the inlets, in this case inlet 1 (FIG. 1), and are assumed to be suspended in solution. Injection of cells into the fluid is not simulated, rather it is assumed that the cells were previously prepared and suspended in the working fluid prior to device activation or mixed into the working fluid in situ using an upstream T- or oscillatory-mixer. In either case, the location of the cells within the channel as they are transported by the working fluid is randomised—only a very small percentage of the cells will be transported along the channel centreline. In the case of healthy red blood cells, a parabolic distribution is expected. Hence, to investigate the ability of the system to successfully trap an arbitrary cell that is transported into the device, the control response was simulated with an initial cell position 15 μm away from the centreline.

FIG. 3 illustrates the efficacy of the control system of FIG. 2 in controlling the position of the cell using fluid flow. Response curves are shown for the control of the y (vertical) position of the cell, vs. time, for case I, case II and case III as defined below. FIG. 3 shows the control response of the cell (the y-position of the cell) as it moves to the saddle point in the centre of the channel for an idealised and two non-ideal cases. Simulation parameters were chosen to model a red blood cell in the flow, where an average red blood cell is assumed, filled predominately with solution similar to water and of radius 4 μm. Relevant parameters for simulation cases I-III are summarised in Table 1 below.

TABLE 1
Parameters chosen for the trapping simulations

| | | | |
|---|---|---|---|
| Simulation Parameter | Case I | Case II | Case III |

| | |
|---|---|
| Imaging rate | 200 fps |
| Cell radius | 4 μm |
| Cell density | 1 (relative to fluid) |
| Initial y location of cell | 15 μm above centre line |
| Proportional gain, x | 0.2 |
| Proportional gain, y | 1.25 |
| Average flow velocity | 1 μms⁻¹ |

| | | | |
|---|---|---|---|
| Observer resolution | Ideal | 0.1 μm | 1 μm |
| Actuator noise | None | None | 1% |
| Actuator time constant | 0 | 50 ms | 100 ms |

As can be seen in FIG. 3 when comparing cases I-III, increasing noise and decreasing observer resolution affects both the stability of the final trap and the time to achieve a stable trap. As resolution decreases and actuator noise increases, oscillatory behaviour becomes more prevalent. However, the overall noise remains low, due to the relative size of the cell and low flow velocities. Even in the low resolution and high noise case (Case III), position noise is much less than 1 μm. This is due to the presence of digitisation noise—the long-term average of the observed location after quantisation will approach the true value, resulting in a stable trap as long as the cell velocities are sufficiently small relative to the acquisition rate. Larger time constants increase the time required to trap the cell, largely due to the increased time required to initially change the flow rates when the control system is activated. An increase of the actuator time constant from 50 ms to 100 ms is sufficient to increase the overall system damping such that the response moves from an underdamped response and begins to approach an overdamped response, further lengthening the trapping time.

Gain optimisation is also addressed by this embodiment. For a number of imaging applications, it is important to minimise oscillation of the cell position. Any small movement of the cell will raise the effective noise floor of the image analysis. Additionally, in situations where a secondary high-resolution camera is used to analyse the cell image, it may be necessary to maintain the cell position within a small region of the channel, as the secondary camera may have a much smaller field of view than the imaging device used for control feedback. In the absence of a closed-form solution for the control response and therefore for the RMS error, non-linear optimisation techniques provide a route to optimisation of the control gains. In this embodiment, the simulation technique is used to map the space of potential solutions and therefore locate the region of lowest RMS error. This kind of optimisation could also be performed online using an adaptive gain estimation technique.

Over 180 simulations were run, varying the proportional gain in the x and y directions. Due to the computational efficiency of the CIFH technique, these simulations took less than 2 hours to complete on a modern multi-core system, corresponding to around 10 CPU-minutes (the product of the number of CPUs and the total runtime of the simulation) per simulation. This contrasts with the time required to generate the CFD data for the parameter space, which was in excess of 42 CPU-days. Therefore, the CIFH technique decreases the simulation time by a factor of more than 500, once the initial dataset has been generated. The control system in each case was configured to stably trap the cell while maintaining a mean flow velocity of 10 μm·s⁻¹ in the inlet channels; after 20 seconds of trapping the cell, the overall flow rates were increased to a mean flow velocity of 100 μm·s⁻¹; the cell was maintained in the trap for a further 20 seconds before the simulation was terminated.

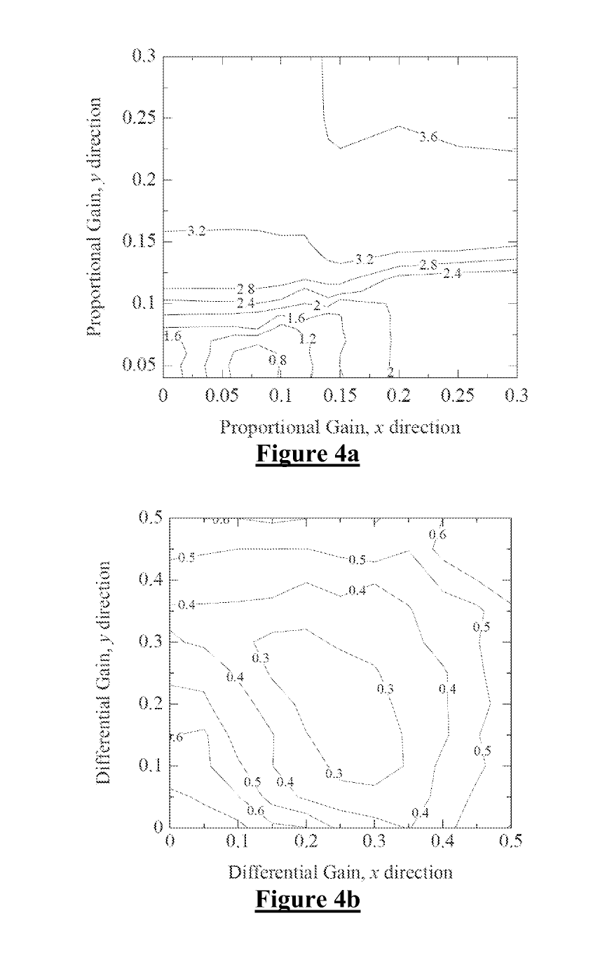

FIG. 4 shows the steady state error norm as a function of various PID gains. The control system sampling rate was fixed at 100 Hz and the flow rate was 100 micrometres per second. In FIG. 4(a) is shown error as a function of proportional gains in the x and y directions. Data for K_{y}<0.04 is not shown as the system was not stable. There is a clear region of stability and low error for 0.05≦Kₓ≦0.12 and 0.04≦K_{y}≦0.08. FIG. 4(b) shows error as a function of differential gains in the x and y directions. For this study the proportional gains were fixed at Kₓ=0.06 and K_{y}=0.06.

FIG. 4a shows a contour of the RMS error of the final 20 seconds (v_{avg}=100 μm·s⁻¹) of the simulation for each solution. Results for proportional gains in the y-direction less than 0.04 are not shown as they did not result in stable cell trap. As expected, there is a combination of proportional gains where the error norm is minimised. Additionally, the error in the y axis is strongly dependent on the gain in the x direction, demonstrating that the control in the two axes cannot be considered linear and separable systems. Further improvement of the steady state error can be achieved with addition of differential gain. FIG. 4b shows the variation of the error with varying differential gains in both directions, with the proportional gains fixed at 0.06. By selecting gains that minimise the error, for example K_{Dx}=0:25, K_{Dy}=0:2 the steady state error norm reduces from 0:75 μm (proportional-only case) to 0:25 μm (proportional and derivative control).

The modelling technique described in the preceding can also be applied to a simple and effective cell sorting device. The geometry of device 100 in FIG. 1 is used to simulate sorting of cells into two groups. A hypothetical device, based on this geometry, with a camera located above the left hand inlet channel (see FIG. 1) is simulated. A number of metrics could be used to identify and categorise the cell, including diameter, shape, or fluorescent response. Using these metrics, the cell would be placed into one of two categories, corresponding to the fluid outlet to which the cell should be directed. For this scheme to be effective, the concentration of the cells in suspension must be low enough so that the fluid forces applied to one cell do not cause the next cell in suspension to flow towards the wrong outlet. The simulator was therefore used to determine the relationship between concentration and accuracy.

A number of simulation runs were performed, with 200 cells sorted in each run. Each cell was arbitrarily assigned a cell type, either alternating between ‘type 0’ and ‘type 1’ or randomly distributed. To simulate a fixed cell identification time, the cell type is not assigned until the cell is 50 μm from the centre of the channel. At the conclusion of each run, the cell type was compared with the outlet port where the cell exited the fluid domain. The percentage of cells that exited through the correct outlet was used as a measure of the sorting success rate. Cells were inserted into the flow by the simulator at a number of different average injection rates. These injection rates were varied with a Gaussian distribution, with a standard deviation of 10% of the mean, to model the uncertainty present in a real dilution or injection scheme. A number of average rates, from one cell every 20 μm up to one cell every 80 μm, were simulated.

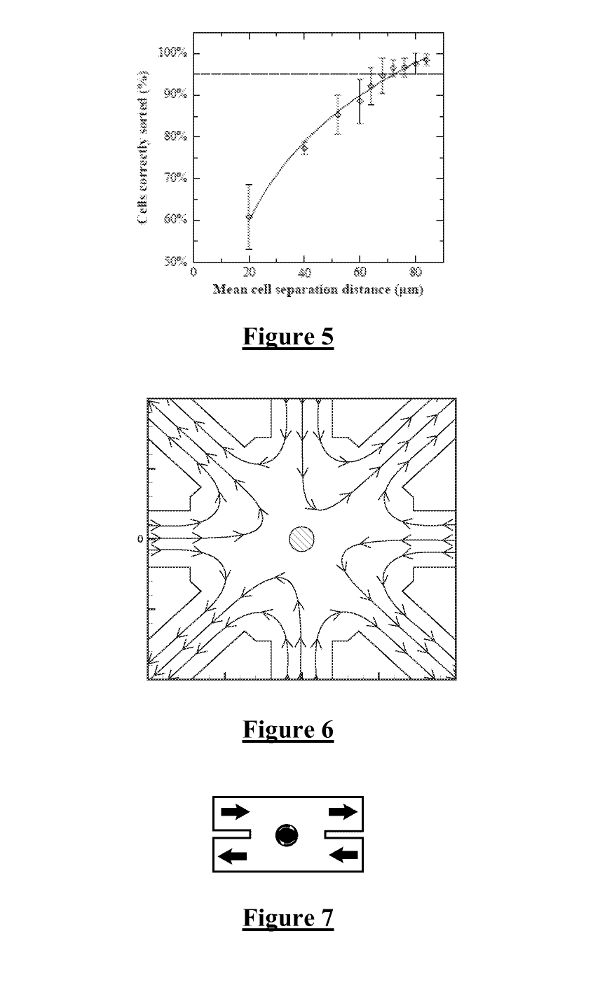

FIG. 5 shows the simulated results of cell sorting using the cross geometry device 100. Cells injected from the left inlet are arbitrarily and randomly assigned a type, and are then directed to the upper or lower outlet based on this type. Here the simulation is used to predict the probability that a given cell, assumed to be correctly identified, is directed to the correct outlet in the channel. Error bars indicate the range of values the tests performed, and the results have been fitted to a logarithmic function (R2=0:99). The average flow velocity at the inlets is 10 μm·s⁻¹. Over 95% accuracy is achieved if the cells are spaced at least 75 μm apart.

The resulting cell sorting success rates are shown in FIG. 5. For each cell concentration, 200 cells were simulated with an alternating distribution of cell types, representing the worst case, and a further 400 cells were simulated with a random distribution, representing the more common case. Excellent accuracy (95%) is achieved with a mean distance between cells of 75 μm and above. By factoring in the expected accuracy of the imaging system in correctly identifying a given cell type, the overall system accuracy of a fluid-based cell sorting design can be estimated. This allows the system to be optimised for the throughput and accuracy demands of a given application.

This embodiment thus provides a fast, efficient and flexible method for simulating control of suspended cells. It has been shown that the cell motion can be decoupled from the flow solver, allowing cell trajectory to be computed independently of the fluid flow field. This allows the flow fields to be computed ahead of time in a computational fluid dynamics solver, dramatically increasing the efficiency of the simulation. For the data presented, the generation of CFD flow fields took 42 CPU-days to complete, whereas a typical control simulation takes under 10 CPU-minutes. Conservatively, the speed-up is well over 500× when compared to a time-stepping CFD method. Additionally, the method is flexible and accurate, as it can take advantage of any flow solver and the error due to interpolation can be minimised by careful choice of the parameter space. Refinement of the parameter grid can be used to improve accuracy where the CFD solutions become non-linear with respect to the control parameters.

It is expected that this method will have wide applicability in the design of new and sophisticated feedback control systems for microchannels. As the control simulation is based on a discrete time stepping method, a large number of existing algorithms and techniques can be simulated using this technique. Additionally, the simple actuator models presented in this embodiment can be extended with measured transfer functions of real actuators and hysteresis effects of the valves to simulate the real world behaviour of complex systems.

This embodiment thus shows that utilising a CIFH control simulation method using a microfluidic cross slot allows active PID control to be used to stabilise and capture a cell in the centre of the microchannel. Optimal gains to minimise error are identified and cell sorting has been investigated and the effective ness of sorting quantified.

FIG. 6 schematically illustrates the port configuration of an eight port device which may be used to simultaneously trap a cell and impart rotation upon the cell. Rotational flow may be induced simultaneously with inducement of a stagnation zone, by asymmetric device construction, or alternatively by tangentially aligned microjets. In an alternative embodiment, the asymmetric construction may comprise four ports, with inlets angularly aligned into the chamber at 12 o'clock and 6 o'clock, and outlets angularly aligned out of the chamber at 1 o'clock and 7 o'clock. Indeed, the outlets may be parallel with the inlets, and FIG. 7 schematically illustrates the configuration of an opposed flow device having a window between two opposed fluid flow paths, which may be used to simultaneously trap a cell and impart rotation upon the cell.

Such an asymmetric construction encourages flow from one port to predominantly flow to the adjacent outlet imparting rotational forces onto the stagnation zone. Reversing the role of inlets and outlets allows the direction of rotation to be reversed. Similar constructions are envisaged with any even number of ports, and can also be envisaged for a device having an odd number of ports, such variations being within the scope of the present invention.

An alternative embodiment uses valves comprising of a voice coil (as an actuator) connected to a pinch device (to convert actuator position to flow resistance). Voice coils are a very fast acting solenoid known to operate with a very low response time and are capable of high acceleration and speed. Their very low response time and fast action gives good high bandwidth control. To improve the accuracy of the proportional voice coil valves, an encoder may be included in combination with a nested feedback control system (whereby one level controls the position of the voice coil, and the higher level controls the flow through the valve).

FIG. 10 is an exploded view of a voice coil actuator assembly of a fluid flow controller, with replaceable fluid tube, in accordance with another embodiment of the invention;

FIG. 11 is a cross sectional view of an 8 port device 1100 in accordance with another embodiment of the invention. The 5^{th} through 8^{th} ports (1122, 1124, 1126, 1128) are aligned substantially normal to a plane in which the other four ports are aligned, to give stabilisation of an object in the vertical axis. Additionally by providing two ports (1122, 1124) above the main plane of the device and two ports below the main plane of the device (1126, 1128), this embodiment provides rotational control of an object trapped between these ports, about an axis which is aligned with the outflow ports.

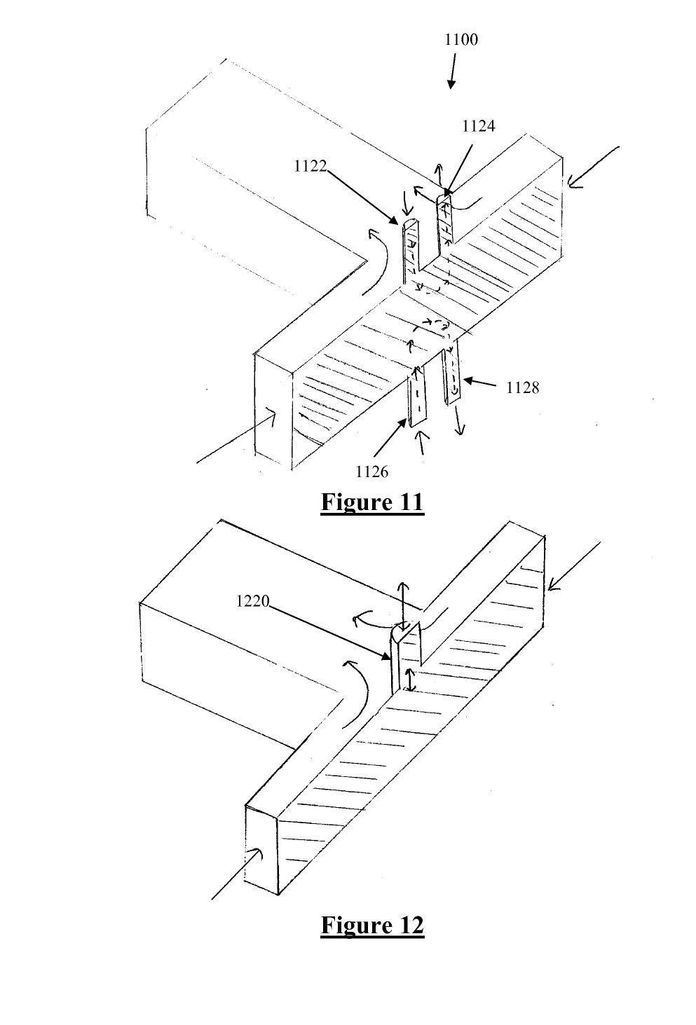

FIG. 12 is a cross sectional view of a 5 port device in accordance with yet another embodiment of the invention. The 5^{th} port 1220 is aligned substantially normal to a plane in which the other four ports are aligned, to give stabilisation of an object in the vertical axis.

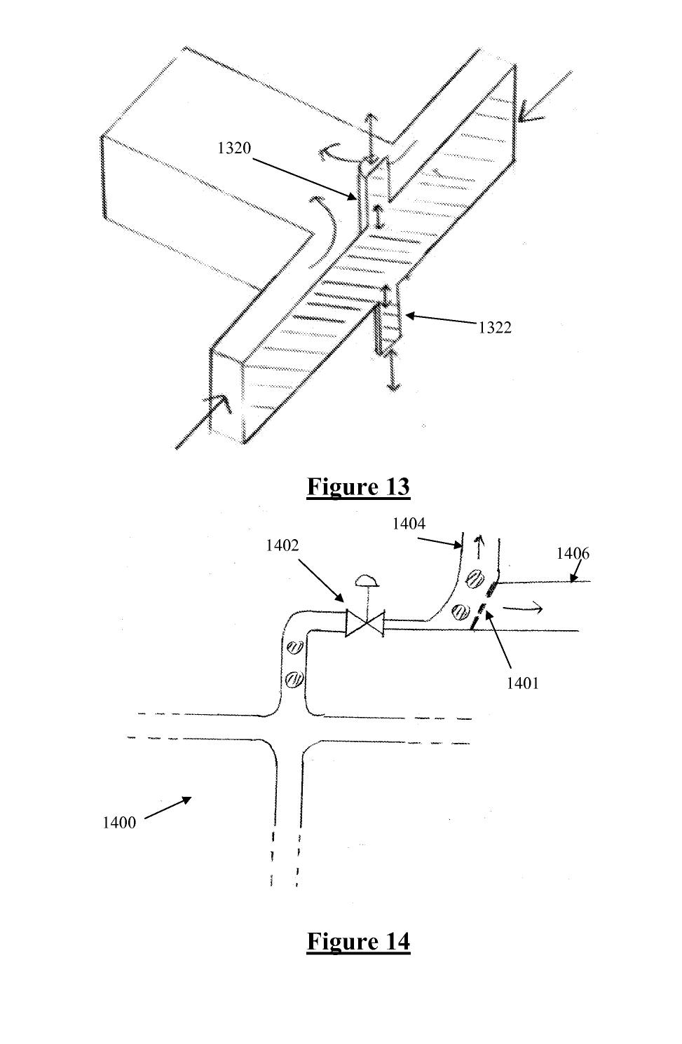

FIG. 13 is a cross sectional view of a 6 port device in accordance with still another embodiment of the invention; The 5^{th} and 6^{th} ports (1320, 1322) are aligned substantially normal to a plane in which the other four ports are aligned, and are above and below that plane respectively, to give improved stabilisation of an object in the vertical axis.

FIG. 14 is a schematic of a 4 port device 1400 in accordance with another embodiment of the invention, having an outlet filter or membrane 1401 and control valve 1402; this provides for a more pure concentration of the cells of interest via output (1404) while the working fluid is passed from outlet (1406). Outlet 1404 could be passed to another filter (not shown) to further improve the purity or concentration of the collected cells of interest.

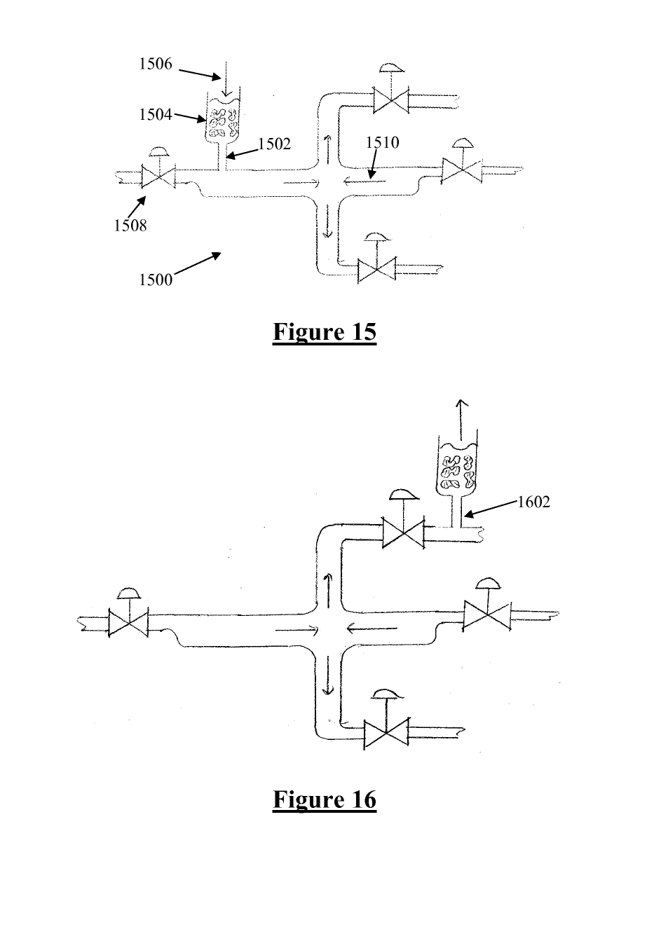

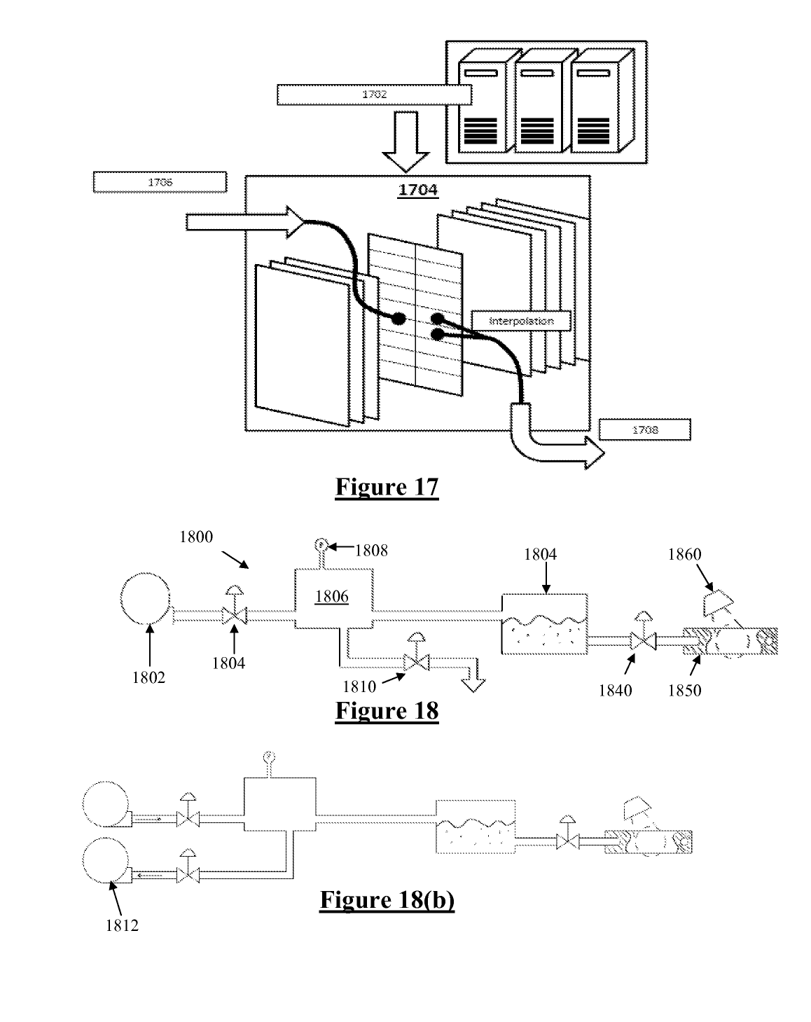

FIG. 15 is a schematic of a 4 port device 1500 in accordance with another embodiment of the invention, having a cell injection inlet 1502 in fluid connection with a suspension of cells (1504) which are controllably subjected to an injection force (1506) such as air pressure or from a nanosyringe. Control valves (1508) control the flow of working fluid as denoted at 1510. The additional port 1502 specifically for the addition or removal of one or more cells, in parallel with the inlets or outlets respectively, is advantageous. With a given concentration of cells and the dosing of a known volume of flow through inlet 1502 a precise number of cells or particles could be added to the solution. An outlet port could work in a similar fashion with suction occurring as the cell is known to pass by that outlet port (as shown at 1602 in FIG. 16). In addition, the outlet 1602 could be used in conjunction with a device to concentrate cells (such as a cyclone) to increase the efficiency of cell removal/suction. The inlet port 1502 and outlet port 1602 may both be provided in another embodiment. Providing such ports decouples the volume of cell samples required from the volume of carrier fluid flow required for device operation, thus permitting the device to be used in relation to very small cell samples.

FIG. 20 illustrates various applications of the device of FIG. 1, including analysis of a cell, a colony, or a tissue sample. FIG. 20 illustrates the generation of compression and stretching forces on the x-y axes of the cell respectively, and shows trapping. Detail A shows the fluid forces for compression and stretching, and it is noted that changing the flow rate allows application of very small stretching and compression forces, for example during device flow rates which are much lower than typical physiological flow rates. Device flow rate can be increased to raise the experienced shear rate to apply the desired stimulus or even to rupture and destroy the cell. FIG. 20 detail B shows a cell captured in an oil droplet which can be useful to better separate the cell from the carrier fluid, if required, and the invention may similarly be used to investigate reagents mixed with water and trapped in an oil medium for example.

These and other embodiments of the invention may thus enable high throughput bench-top devices that can measure properties of single RBCs, enabling large scale assays. A device capable of determining elastic and viscoelastic properties of cells at a high rate of throughput, will mark a significant shift in the research towards improved treatments for disease, as well as in the point-of-care diagnosis of these diseases. The novel combination of micro-fluidic flows and imaging technologies allows this invention to exploit recent advances in both lab-on-a-chip and real-time control technology to design and build a device whereby cells are acted on by the fluid forces in a lab-chip environment for example to effect a lab-on-a-chip device for the measurement of cell mechanics.

It will be appreciated by persons skilled in the art that numerous variations and/or modifications may be made to the invention as shown in the specific embodiments without departing from the spirit or scope of the invention as broadly described. For example, some embodiments of the invention may be applied in assessment of non-blood borne cells such as oocytes or the multicellular early embryo. The outer membrane of the oocyte, the zona pellucida, shows a degree of flexibility and the success of assisted reproduction techniques such as intracellular sperm injection (ICSI) are thought to be related to the degree of zona pellucida flexibility. Embodiments of this invention may thus provide an atraumatic method to sort embryos on the basis of zona pellucida flexibility. Further, in the embodiments shown in the Figures, where a valve is shown it is to be appreciated that an alternative embodiment is to instead use a fluid flow controller such as is shown in FIGS. 8 and/or 18. Moreover, the biocompatible pumps may additionally or alternatively each comprise a high precision linear stepper motor controller, each stepper motor controller being coupled with a high precision stepper motor pinch valve and tube assembly and pressure sensor.

The present embodiments are, therefore, to be considered in all respects as illustrative and not restrictive.

1. A method of determining biomechanical properties of a cell, the method comprising:
introducing the cell into a multiport flow device, the device being configured such that during fluid flow at least one stagnation zone arises in an expected location within the device;
trapping the cell in the stagnation zone of the device;
applying a selected physical stimulus to the cell; and
observing the cell while trapped to detect an effect of the applied physical stimulus and to thereby determine biomechanical properties of the cell.

2. The method of claim 1 wherein the cell is a red blood cell and the biomechanical property is the stiffness of the red blood cell as determined in response to the selected physical stimulus.

3. The method of claim 2, further comprising giving a diagnosis of a disease comprising one of diabetes, cancer, obesity and malaria.

4. A device for determining biomechanical properties of a cell, the device comprising:
a plurality of fluid ports communicating with a fluid chamber, the ports and chamber being configured such that during fluid flow at least one stagnation zone arises in an expected location within the device; and
a fluid flow controller for trapping the cell in the stagnation zone of the device and, while the cell is trapped, for applying fluid forces to the cell so as to apply a selected physical stimulus to the cell; and
a detector for observing the cell while trapped to detect an effect of the applied physical stimulus and to thereby determine biomechanical properties of the cell.

5. A method of sorting a cell, the method comprising:
determining biomechanical properties of the cell by:
introducing the cell into a multiport flow device, the device being configured such that during fluid flow at least one stagnation zone arises in an expected location within the device;
trapping the cell in the stagnation zone of the device;
applying a selected physical stimulus to the cell; and
observing the cell while trapped to detect an effect of the applied physical stimulus and to thereby determine biomechanical properties of the cell; and wherein the multiport flow device comprises at least first and second fluid outlets based on the determined biomechanical property of the cell, selecting an outlet to which the cell should be directed; and
controlling fluid flow within the device to alter the location of the stagnation zone and to direct the cell to the selected outlet.

6. The method according to claim 5, comprising passing the cell through a cascade of sorting stages each comprising a multiport flow device configured to sort cells based on detecting a unique property or property-set of the cell.

7. A device for trapping a particle, the device comprising:
a chamber in fluid communication with at least two fluid inlets and at least two fluid outlets, the device being configured such that during fluid flow at least one stagnation zone arises in an expected location within the chamber, within which a particle may be captured and observed; and
the device comprising a means to introduce a component of rotational fluid flow within the chamber such that rotational forces can be applied to a particle captured within the stagnation zone.

8. The device of claim 7, wherein the fluid controller is further configured to control fluid flow through the inlets and outlets in a manner to apply stretching forces to a captured particle by increasing inlet flow into the chamber through opposing inlets and providing reduced inlet flow through other inlets, to apply simultaneous rotation and stretching forces to a captured particle.

9. The device of claim 7 further comprising at least one port above and/or below the nominal plane.

10. The device of claim 11, having at least one port above the nominal plane and at least one port below the nominal plane, and wherein the fluid controllers are further configured to apply compression to a captured particle by simultaneously causing fluid flow into the device through the out-of-plane ports, and/or to apply stretching to a captured particle by simultaneously causing fluid flow out of the device through the out-of-plane ports.

11. The device of claim 4 wherein
each inlet and each outlet are positioned substantially within a single nominal plane; and
at least one out-of-plane inlet or outlet whereby control of fluid flow through the out-of-plane inlet or outlet allows a captured particle to be moved towards or away from the nominal plane.

12. A method of characterising fluid conditions which cause platelet activation, the method comprising:
passing a platelet through a fluid path;
controllably altering flow rate and fluid pressure within the fluid path in order to subject the platelet to a shear rate which is time varying and which has a selected profile over time during passage of the platelet through the flow path; and
detecting whether the platelet activates in response to the shear rate profile experienced.

13. A method of determining biomechanical properties of a cell, the method comprising:
introducing the cell into a multiport flow device, the device being configured such that during fluid flow at least one stagnation zone arises in an expected location within the device;
trapping the cell in the stagnation zone of the device;
first observing the cell while trapped, to determine initial biomechanical properties of the cell;
after the first observing, applying a selected stimulus to the cell; and
after the stimulus, observing the cell a second time while trapped to determine subsequent biomechanical properties of the cell caused by the effect of the applied stimulus upon the cell.

14. The method of claim 13, further comprising observing the cell during application of the stimulus in order to measure transient effects of the stimulus.

15. The method of claim 13, wherein the initial biomechanical properties of the cell, the transient response of the cell, and the subsequent biomechanical properties of the cell are all obtained, and further comprising determining both cell viscosity and membrane stiffness of the cell.

16. The method of claim 13, further comprising obtaining a measure of cell behaviour during tank treading.

17. The method of claim 1 wherein the physical stimulus comprises a static or time varying stretching force applied by fluid control.

18. The method of claim 1 wherein the physical stimulus comprises a static or time varying rotational force about one or more axes.

19. The method of claim 1 wherein the physical stimulus comprises a static or time varying shear rate applied to the trapped cell.

20. The method of claim 1 wherein the physical stimulus comprises a static or time varying pressure.

21. The method of claim 1 wherein the physical stimulus comprises a static or time varying acceleration caused by moving the stagnation point and trapped particle.

22. The method of claim 1 wherein the observing comprises imaging the cell.

23. A method for diagnosing or assessing the stage of a disease or disorder in a subject, comprising:
obtaining a cell sample from the subject;
determining a biomechanical property of the cell by:
introducing the cell into a multiport flow device, the device being configured such that during fluid flow at least one stagnation zone arises in an expected location within the device;
trapping the cell in the stagnation zone of the device;
applying a selected physical stimulus to the cell; and
observing the cell while trapped to detect an effect of the applied physical stimulus and to thereby determine biomechanical properties of the cell; and
comparing the biomechanical property of the cell to a normal control of the same cell type as the cell sample from the subject to detect evidence of the disease or disorder.

24. A method for diagnosing or assessing the stage of a disease or disorder in a subject, comprising:
obtaining a cell sample from the subject;
determining biomechanical properties of the cell by:
introducing the cell into a multiport flow device, the device being configured such that during fluid flow at least one stagnation zone arises in an expected location within the device;
trapping the cell in the stagnation zone of the device;
first observing the cell while trapped, to determine initial biomechanical properties of the cell;
after the first observing, applying a selected stimulus to the cell; and
after the stimulus, observing the cell a second time while trapped to determine subsequent biomechanical properties of the cell caused by the effect of the applied stimulus upon the cell;
comparing the biomechanical properties of the cell to a normal control of the same cell type as the cell sample from the subject to detect evidence of the disease or disorder.

25. The method of claim 23, wherein the biomechanical property is selected from the group consisting of the shear modulus, the viscosity and the apparent bending stiffness of the cell's membrane.

26. The method of claim 24, wherein the biomechanical properties are selected from the group consisting of the shear modulus, the viscosity and the apparent bending stiffness of the cell's membrane.

27. The method of claim 23, wherein the cell sample is selected from the group consisting of peripheral blood, body fluid, and tissue.

28. The method of claim 27, wherein the cell sample is red blood cells.

29. The method of claim 27, wherein the tissue is selected from the group consisting of breast, bladder, male reproductive system, female reproductive system, bone, pancreas, brain, skin, digestive tract and lung tissues.

30. The method of claim 23, where the disease or disorder is selected from the group consisting of diabetes, cancer and malaria.

31. The method of claim 30, wherein the cell is diagnostic of a pre-invasive cancer.

32. The method of claim 30, wherein the cell is diagnostic of an invasive cancer.

33. A method of monitoring a response to a therapy comprising performing the method of claim 23 on a cell sample from a subject to whom the therapy has been administered.

34. A method for monitoring a subject for a disease or disorder comprising performing the method of claim 23 on a cell sample from the subject.

35. A method for selecting a subject for a therapy directed to a disease or disorder comprising performing the method of claim 23 on a cell sample from the subject.

36. The method of claim 35, wherein the therapy is selected from the group consisting anti-neoplastic therapy, antibiotic therapy, prophylactic drugs, lifestyle modification, vaccine therapy, biologic therapy and anti-angiogenic therapy.

37. The method of claim 23, further comprising planning a course of further diagnostic testing and treatment.

38. A method of determining properties of a micro-particle, the method comprising:
introducing the micro-particle into a multiport flow device, the device being configured such that during fluid flow at least one stagnation zone arises in an expected location within the device;
trapping the micro-particle in the stagnation zone of the device by simultaneously monitoring particle position and measuring and controlling the fluid flow field in the stagnation zone; and
observing at least one behaviour of the micro-particle while trapped and determining at least one property of the micro-particle from the or each observed behaviour by reference to a micro-particle model.

39. A microfluidics fluid flow control system comprising:
a deformable member defining a microfluidic passage, whereby deformation of the member alters a cross section of the passage and the flow resistance of the passage;
a pressure source in fluid communication with the deformable passage;
an actuator for controllably causing deformation of the deformable member so as to controllably occlude the passage;
an actuator controller which linearly actuates the actuator, the actuator controller operating in response to an input signal indicating a desired fluid flow through the passage and in response to a feedback signal representing an observed feedback variable.

40. The system of claim 39 wherein the actuator is the armature of a solenoid controlled by the actuator controller.

41. The system of claim 39 wherein the feedback variable comprises an observed fluid flow rate through the passage.

42. The system of claim 39 wherein the observed feedback variable comprises an observed actuator position, as measured by an actuator position meter.

43. The system of claim 39 wherein the observed feedback variable comprises an observed pressure drop across the occlusion.

44. (canceled)

45. (canceled)
